SEQUENCE LISTING (192 sequences)

SEQ ID NO: 1 - DNA, 58 bp, Artificial sequence
  synthetic sequence
ccggctgggagaagttcgtgtatttctcgagaaatacacgaacttctcccagtttttg

SEQ ID NO: 2 - DNA, 58 bp, Artificial sequence
  synthetic sequence
ccgggcccttaaccatcgagataaactcgagtttatctcgatggttaagggctttttg

SEQ ID NO: 3 - DNA, 58 bp, Artificial sequence
  synthetic sequence
ccggtgcccttaaccatcgagataactcgagttatctcgatggttaagggcatttttg

SEQ ID NO: 4 - DNA, 58 bp, Artificial sequence
  synthetic sequence
ccgggcatgtaaacacgcctgcaaactcgagtttgcaggcgtgtttacatgctttttg

SEQ ID NO: 5 - DNA, 58 bp, Artificial sequence
  synthetic sequence
ccgggcgcttagtgtaccacatcaactcgagttgatgtggtacactaagcgctttttg

SEQ ID NO: 6 - DNA, 65 bp, Artificial sequence
  synthetic sequence
tactatgcctgtgtcttctccaccacgcttcaagagagcgtggtggagaagacacaggcatagta

SEQ ID NO: 7 - DNA, 65 bp, Artificial sequence
  synthetic sequence
ctggatgatggtcttcagcctctttctgttcaagagacagaaagaggctgaagaccatcatccag

SEQ ID NO: 8 - DNA, 65 bp, Artificial sequence
  synthetic sequence
ggaggaatggacagacagaggatgagctctcaagaggagctcatcctctgtctgtccattcctcc

SEQ ID NO: 9 - DNA, 65 bp, Artificial sequence
  synthetic sequence
tccatttggcagagcttcttccaggtgcctcaagagggcacctggaagaagctctgccaaatgga

SEQ ID NO: 10 - DNA, 68 bp, Artificial sequence
  synthetic sequence
agatctggtaatgattgattaaatatacctgacccatatatttaatcaatcattacctttttggtacc

SEQ ID NO: 11 - DNA, 68 bp, Artificial sequence
  synthetic sequence
agatctgtattcatggatttatttatacctgacccatataaataaatccatgaatactttttggtacc

SEQ ID NO: 12 - DNA, 68 bp, Artificial sequence
  synthetic sequence
agatctgatattcatggatttattatacctgacccatataataaatccatgaatatctttttggtacc

SEQ ID NO: 13 - DNA, 68 bp, Artificial sequence
  synthetic sequence
agatctgcatggatttatttatttatacctgacccatataaataaataaatccatgctttttggtacc

SEQ ID NO: 14 - DNA, 68 bp, Artificial sequence
  synthetic sequence
agatctgattcatggatttatttaatacctgacccatattaaataaatccatgaatctttttggtacc

SEQ ID NO: 15 - DNA, 68 bp, Artificial sequence
  synthetic sequence
agatctgaatattcatggatttatatacctgacccatatataaatccatgaatattctttttggtacc

SEQ ID NO: 16 - DNA, 68 bp, Artificial sequence
  synthetic sequence
agatctgttgtaatgattgattaaatacctgacccatatttaatcaatcattacaactttttggtacc

SEQ ID NO: 17 - DNA, 68 bp, Artificial sequence
  synthetic sequence
agatctgcaagatagtttatctaaatacctgacccatatttagataaactatcttgctttttggtacc

SEQ ID NO: 18 - DNA, 68 bp, Artificial sequence
  synthetic sequence
agatctggagaagttcgtgtatttatacctgacccatataaatacacgaacttctcctttttggtacc

SEQ ID NO: 19 - DNA, 68 bp, Artificial sequence
  synthetic sequence
agatctgtaatgattgattaaatgatacctgacccatatcatttaatcaatcattactttttggtacc

SEQ ID NO: 20 - DNA, 68 bp, Artificial sequence
  synthetic sequence
agatctgtttgtaatgattgattaatacctgacccatattaatcaatcattacaaactttttggtacc

SEQ ID NO: 21 - DNA, 68 bp, Artificial sequence
  synthetic sequence
agatctgttcatggatttatttatatacctgacccatatataaataaatccatgaactttttggtacc

SEQ ID NO: 22 - DNA, 68 bp, Artificial sequence
  synthetic sequence
agatctggattaaatgcaagatagatacctgacccatatctatcttgcatttaatcctttttggtacc

SEQ ID NO: 23 - DNA, 68 bp, Artificial sequence
  synthetic sequence
agatctgtcatggatttatttattatacctgacccatataataaataaatccatgactttttggtacc

SEQ ID NO: 24 - DNA, 68 bp, Artificial sequence
  synthetic sequence
agatctgaaatattcatggatttaatacctgacccatattaaatccatgaatatttctttttggtacc

SEQ ID NO: 25 - DNA, 68 bp, Artificial sequence
  synthetic sequence
agatctgattaaatgcaagatagtatacctgacccatatactatcttgcatttaatctttttggtacc

SEQ ID NO: 26 - DNA, 68 bp, Artificial sequence
  synthetic sequence
agatctgtgtatgtggcatgaataatacctgacccatattattcatgccacatacactttttggtacc

SEQ ID NO: 27 - DNA, 68 bp, Artificial sequence
  synthetic sequence
agatctggcaagatagtttatctaatacctgacccatattagataaactatcttgcctttttggtacc

SEQ ID NO: 28 - DNA, 68 bp, Artificial sequence
  synthetic sequence
agatctgtaaatgcaagatagtttatacctgacccatataaactatcttgcatttactttttggtacc

SEQ ID NO: 29 - DNA, 68 bp, Artificial sequence
  synthetic sequence
agatctgaatgcaagatagtttatatacctgacccatatataaactatcttgcattctttttggtacc

SEQ ID NO: 30 - skipped

SEQ ID NO: 31 - DNA, 20 bp, Artificial sequence
  synthetic sequence
cgagtgcccgacactcgggg

SEQ ID NO: 32 - DNA, 20 bp, Artificial sequence
  synthetic sequence
tgtaacctatcctttatctg

SEQ ID NO: 33 - DNA, 20 bp, Artificial sequence
  synthetic sequence
tgcctctcagattgacggcg

SEQ ID NO: 34 - DNA, 20 bp, Artificial sequence
  synthetic sequence
agttacgggtgctcttgcgt

SEQ ID NO: 35 - DNA, 20 bp, Artificial sequence
  synthetic sequence
gcgcttagtgtaccacatca

SEQ ID NO: 36 - DNA, 20 bp, Artificial sequence
  synthetic sequence
aggatcggtatcgtccatca

SEQ ID NO: 37 - DNA, 20 bp, Artificial sequence
  synthetic sequence
ctggctgcccttaaccatcg

SEQ ID NO: 38 - DNA, 20 bp, Artificial sequence
  synthetic sequence
atggccgagtgcccgacact

SEQ ID NO: 39 - DNA, 20 bp, Artificial sequence
  synthetic sequence
ggccgagtgcccgacactcg

SEQ ID NO: 40 - DNA, 20 bp, Artificial sequence
  synthetic sequence
gggtagagctgccacatgat

SEQ ID NO: 41 - DNA, 20 bp, Artificial sequence
  synthetic sequence
caagcctgctgcctatcatg

SEQ ID NO: 42 - DNA, 20 bp, Artificial sequence
  synthetic sequence
gttacgggtgctcttgcgtc

SEQ ID NO: 43 - DNA, 20 bp, Artificial sequence
  synthetic sequence
gtcgggaagacgtcgtcctg

SEQ ID NO: 44 - DNA, 20 bp, Artificial sequence
  synthetic sequence
ctctcagattgacggcgtgg

SEQ ID NO: 45 - DNA, 20 bp, Artificial sequence
  synthetic sequence
gggaagacgtcgtcctgggg

SEQ ID NO: 46 - DNA, 20 bp, Artificial sequence
  synthetic sequence
gaggatcggtatcgtccatc

SEQ ID NO: 47 - DNA, 20 bp, Artificial sequence
  synthetic sequence
caaatacacgaacttctccc

SEQ ID NO: 48 - DNA, 20 bp, Artificial sequence
  synthetic sequence
aaaggataggttacagttac

SEQ ID NO: 49 - DNA, 20 bp, Artificial sequence
  synthetic sequence
tggctgtacctcgatggtta

SEQ ID NO: 50 - DNA, 20 bp, Artificial sequence
  synthetic sequence
cgtcgggaagacgtcgtcct

SEQ ID NO: 51 - DNA, 20 bp, Artificial sequence
  synthetic sequence
agaaactggagtctgaggat

SEQ ID NO: 52 - DNA, 20 bp, Artificial sequence
  synthetic sequence
gctgccacatgataggcagc

SEQ ID NO: 53 - DNA, 20 bp, Artificial sequence
  synthetic sequence
caaagctggctgtacctcga

SEQ ID NO: 54 - DNA, 20 bp, Artificial sequence
  synthetic sequence
gagtgtcgggcactcggcca

SEQ ID NO: 55 - DNA, 20 bp, Artificial sequence
  synthetic sequence
ggctgtacctcgatggttaa

SEQ ID NO: 56 - DNA, 20 bp, Artificial sequence
  synthetic sequence
ctcaccatacatcagtcatc

SEQ ID NO: 57 - DNA, 20 bp, Artificial sequence
  synthetic sequence
gcgtcgggaagacgtcgtcc

SEQ ID NO: 58 - DNA, 20 bp, Artificial sequence
  synthetic sequence
ggaggcagtcaccgaccacc

SEQ ID NO: 59 - DNA, 20 bp, Artificial sequence
  synthetic sequence
gaaagacactcaccttgatg

SEQ ID NO: 60 - DNA, 20 bp, Artificial sequence
  synthetic sequence
gtacactaagcgccagaaac

SEQ ID NO: 61 - DNA, 20 bp, Artificial sequence
  synthetic sequence
acttctcccaggcccacagg

SEQ ID NO: 62 - DNA, 20 bp, Artificial sequence
  synthetic sequence
gtgactgcctccccgagtgt

SEQ ID NO: 63 - DNA, 20 bp, Artificial sequence
  synthetic sequence
ctccccgagtgtcgggcact

SEQ ID NO: 64 - DNA, 20 bp, Artificial sequence
  synthetic sequence
gcgccagaaactggagtctg

SEQ ID NO: 65 - DNA, 20 bp, Artificial sequence
  synthetic sequence
tggacgagaagcagcacgcc

SEQ ID NO: 66 - DNA, 20 bp, Artificial sequence
  synthetic sequence
aggcccacaggcggtccggg

SEQ ID NO: 67 - DNA, 20 bp, Artificial sequence
  synthetic sequence
atgtggcagctctaccctga

SEQ ID NO: 68 - DNA, 20 bp, Artificial sequence
  synthetic sequence
aggcttgggcctatctgggt

SEQ ID NO: 69 - DNA, 20 bp, Artificial sequence
  synthetic sequence
tggccgagtgcccgacactc

SEQ ID NO: 70 - DNA, 20 bp, Artificial sequence
  synthetic sequence
acatgataggcagcaggctt

SEQ ID NO: 71 - DNA, 20 bp, Artificial sequence
  synthetic sequence
ccgaccacccggaccgcctg

SEQ ID NO: 72 - DNA, 20 bp, Artificial sequence
  synthetic sequence
cctatgacccccacccagat

SEQ ID NO: 73 - DNA, 20 bp, Artificial sequence
  synthetic sequence
tgactgcctccccgagtgtc

SEQ ID NO: 74 - DNA, 20 bp, Artificial sequence
  synthetic sequence
cgaccacccggaccgcctgt

SEQ ID NO: 75 - DNA, 20 bp, Artificial sequence
  synthetic sequence
ctgggagaagttcgtgtatt

SEQ ID NO: 76 - DNA, 20 bp, Artificial sequence
  synthetic sequence
tgggggtcataggcctcccc

SEQ ID NO: 77 - DNA, 20 bp, Artificial sequence
  synthetic sequence
cgaacttctcccaggcccac

SEQ ID NO: 78 - DNA, 20 bp, Artificial sequence
  synthetic sequence
taaaggataggttacagtta

SEQ ID NO: 79 - DNA, 20 bp, Artificial sequence
  synthetic sequence
ccacaggcggtccgggtggt

SEQ ID NO: 80 - DNA, 20 bp, Artificial sequence
  synthetic sequence
tcccaggcccacaggcggtc

SEQ ID NO: 81 - DNA, 20 bp, Artificial sequence
  synthetic sequence
ctcgatggttaagggcagcc

SEQ ID NO: 82 - DNA, 20 bp, Artificial sequence
  synthetic sequence
cagcaggcttgggcctatct

SEQ ID NO: 83 - DNA, 20 bp, Artificial sequence
  synthetic sequence
gcttgggcctatctgggtgg

SEQ ID NO: 84 - DNA, 20 bp, Artificial sequence
  synthetic sequence
ggcttgggcctatctgggtg

SEQ ID NO: 85 - DNA, 20 bp, Artificial sequence
  synthetic sequence
cttctcctcagataaaggat

SEQ ID NO: 86 - DNA, 20 bp, Artificial sequence
  synthetic sequence
cccaggcccacaggcggtcc

SEQ ID NO: 87 - DNA, 20 bp, Artificial sequence
  synthetic sequence
caggcttgggcctatctggg

SEQ ID NO: 88 - DNA, 20 bp, Artificial sequence
  synthetic sequence
cctatctgggtgggggtcat

SEQ ID NO: 89 - DNA, 20 bp, Artificial sequence
  synthetic sequence
ctctctgcctctcagattga

SEQ ID NO: 90 - DNA, 20 bp, Artificial sequence
  synthetic sequence
cccggaccgcctgtgggcct

SEQ ID NO: 91 - DNA, 20 bp, Artificial sequence
  synthetic sequence
gatcctcagactccagtttc

SEQ ID NO: 92 - DNA, 20 bp, Artificial sequence
  synthetic sequence
cacatgataggcagcaggct

SEQ ID NO: 93 - DNA, 20 bp, Artificial sequence
  synthetic sequence
acccggaccgcctgtgggcc

SEQ ID NO: 94 - DNA, 20 bp, Artificial sequence
  synthetic sequence
gcagcaggcttgggcctatc

SEQ ID NO: 95 - DNA, 20 bp, Artificial sequence
  synthetic sequence
ctccccgagtgtcgggcact

SEQ ID NO: 96 - DNA, 20 bp, Artificial sequence
  synthetic sequence
caaatacacgaacttctccc

SEQ ID NO: 97 - DNA, 20 bp, Artificial sequence
  synthetic sequence
caaagctggctgtacctcga

SEQ ID NO: 98 - DNA, 20 bp, Artificial sequence
  synthetic sequence
cttctcctcagataaaggat

SEQ ID NO: 99 - DNA, 20 bp, Artificial sequence
  synthetic sequence
gttacgggtgctcttgcgtc

SEQ ID NO: 100 - DNA, 20 bp, Artificial sequence
  synthetic sequence
ggtggcggccgcatgctgcc

SEQ ID NO: 101 - DNA, 20 bp, Artificial sequence
  synthetic sequence
ctttatatccgggcagcatg

SEQ ID NO: 102 - DNA, 20 bp, Artificial sequence
  synthetic sequence
cccgccccgccgcttggtgg

SEQ ID NO: 103 - DNA, 20 bp, Artificial sequence
  synthetic sequence
agcatgcggccgccaccaag

SEQ ID NO: 104 - DNA, 20 bp, Artificial sequence
  synthetic sequence
cctcccgccccgccgcttgg

SEQ ID NO: 105 - DNA, 20 bp, Artificial sequence
  synthetic sequence
atgcggccgccaccaagcgg

SEQ ID NO: 106 - DNA, 20 bp, Artificial sequence
  synthetic sequence
cgtcctcccgccccgccgct

SEQ ID NO: 107 - DNA, 20 bp, Artificial sequence
  synthetic sequence
tgcggccgccaccaagcggc

SEQ ID NO: 108 - DNA, 20 bp, Artificial sequence
  synthetic sequence
gcggccgccaccaagcggcg

SEQ ID NO: 109 - DNA, 20 bp, Artificial sequence
  synthetic sequence
gccgccaccaagcggcgggg

SEQ ID NO: 110 - DNA, 20 bp, Artificial sequence
  synthetic sequence
ccgccaccaagcggcggggc

SEQ ID NO: 111 - DNA, 20 bp, Artificial sequence
  synthetic sequence
ccaccaagcggcggggcggg

SEQ ID NO: 112 - DNA, 20 bp, Artificial sequence
  synthetic sequence
caagcggcggggcgggagga

SEQ ID NO: 113 - DNA, 20 bp, Artificial sequence
  synthetic sequence
gcggggcgggaggacggcct

SEQ ID NO: 114 - DNA, 20 bp, Artificial sequence
  synthetic sequence
cggggcgggaggacggcctt

SEQ ID NO: 115 - DNA, 20 bp, Artificial sequence
  synthetic sequence
ggggcgggaggacggccttg

SEQ ID NO: 116 - DNA, 20 bp, Artificial sequence
  synthetic sequence
gcgggaggacggccttgggg

SEQ ID NO: 117 - DNA, 20 bp, Artificial sequence
  synthetic sequence
cgacccggggtcccgcccca

SEQ ID NO: 118 - DNA, 20 bp, Artificial sequence
  synthetic sequence
cgggaggacggccttggggc

SEQ ID NO: 119 - DNA, 20 bp, Artificial sequence
  synthetic sequence
acggccttggggcgggaccc

SEQ ID NO: 120 - DNA, 20 bp, Artificial sequence
  synthetic sequence
cggccttggggcgggacccc

SEQ ID NO: 121 - DNA, 20 bp, Artificial sequence
  synthetic sequence
cttggggcgggaccccgggt

SEQ ID NO: 122 - DNA, 20 bp, Artificial sequence
  synthetic sequence
aaggtcgttctcccgacccg

SEQ ID NO: 123 - DNA, 20 bp, Artificial sequence
  synthetic sequence
ttggggcgggaccccgggtc

SEQ ID NO: 124 - DNA, 20 bp, Artificial sequence
  synthetic sequence
caaggtcgttctcccgaccc

SEQ ID NO: 125 - DNA, 20 bp, Artificial sequence
  synthetic sequence
ccaaggtcgttctcccgacc

SEQ ID NO: 126 - DNA, 20 bp, Artificial sequence
  synthetic sequence
ccgggtcgggagaacgacct

SEQ ID NO: 127 - DNA, 20 bp, Artificial sequence
  synthetic sequence
cgggtcgggagaacgacctt

SEQ ID NO: 128 - DNA, 20 bp, Artificial sequence
  synthetic sequence
gggtcgggagaacgaccttg

SEQ ID NO: 129 - DNA, 20 bp, Artificial sequence
  synthetic sequence
tcgggagaacgaccttgggg

SEQ ID NO: 130 - DNA, 20 bp, Artificial sequence
  synthetic sequence
ctgccttacgccccgcccca

SEQ ID NO: 131 - DNA, 20 bp, Artificial sequence
  synthetic sequence
cgggagaacgaccttggggc

SEQ ID NO: 132 - DNA, 20 bp, Artificial sequence
  synthetic sequence
gggagaacgaccttggggcg

SEQ ID NO: 133 - DNA, 20 bp, Artificial sequence
  synthetic sequence
cgaccttggggcggggcgta

SEQ ID NO: 134 - DNA, 20 bp, Artificial sequence
  synthetic sequence
cttggggcggggcgtaaggc

SEQ ID NO: 135 - DNA, 20 bp, Artificial sequence
  synthetic sequence
gggcggggcgtaaggcagga

SEQ ID NO: 136 - DNA, 20 bp, Artificial sequence
  synthetic sequence
ggcggggcgtaaggcaggat

SEQ ID NO: 137 - DNA, 20 bp, Artificial sequence
  synthetic sequence
gcggggcgtaaggcaggatg

SEQ ID NO: 138 - DNA, 20 bp, Artificial sequence
  synthetic sequence
gggcgtaaggcaggatgggg

SEQ ID NO: 139 - DNA, 20 bp, Artificial sequence
  synthetic sequence
ggcgtaaggcaggatggggc

SEQ ID NO: 140 - DNA, 20 bp, Artificial sequence
  synthetic sequence
gcgtaaggcaggatggggcg

SEQ ID NO: 141 - DNA, 20 bp, Artificial sequence
  synthetic sequence
gcaggatggggcggggtttg

SEQ ID NO: 142 - DNA, 20 bp, Artificial sequence
  synthetic sequence
caggatggggcggggtttgt

SEQ ID NO: 143 - DNA, 20 bp, Artificial sequence
  synthetic sequence
aggatggggcggggtttgtg

SEQ ID NO: 144 - DNA, 20 bp, Artificial sequence
  synthetic sequence
ggatggggcggggtttgtgg

SEQ ID NO: 145 - DNA, 20 bp, Artificial sequence
  synthetic sequence
ggcggggtttgtgggggtct

SEQ ID NO: 146 - DNA, 20 bp, Artificial sequence
  synthetic sequence
ggggtttgtgggggtcttgg

SEQ ID NO: 147 - DNA, 20 bp, Artificial sequence
  synthetic sequence
gggggtcttggtggcaagtg

SEQ ID NO: 148 - DNA, 20 bp, Artificial sequence
  synthetic sequence
ggggtcttggtggcaagtga

SEQ ID NO: 149 - DNA, 20 bp, Artificial sequence
  synthetic sequence
ggcaagtgagggtcccgcgc

SEQ ID NO: 150 - DNA, 20 bp, Artificial sequence
  synthetic sequence
ccgggacgtagcgcctgcgc

SEQ ID NO: 151 - DNA, 20 bp, Artificial sequence
  synthetic sequence
gccgggacgtagcgcctgcg

SEQ ID NO: 152 - DNA, 20 bp, Artificial sequence
  synthetic sequence
cccgcgcaggcgctacgtcc

SEQ ID NO: 153 - DNA, 20 bp, Artificial sequence
  synthetic sequence
tccagggagcaagtcaggcc

SEQ ID NO: 154 - DNA, 20 bp, Artificial sequence
  synthetic sequence
ttccagggagcaagtcaggc

SEQ ID NO: 155 - DNA, 20 bp, Artificial sequence
  synthetic sequence
accgttccagggagcaagtc

SEQ ID NO: 156 - DNA, 20 bp, Artificial sequence
  synthetic sequence
tcccggcctgacttgctccc

SEQ ID NO: 157 - DNA, 20 bp, Artificial sequence
  synthetic sequence
gcctgacttgctccctggaa

SEQ ID NO: 158 - DNA, 20 bp, Artificial sequence
  synthetic sequence
caggagctgccaccgttcca

SEQ ID NO: 159 - DNA, 20 bp, Artificial sequence
  synthetic sequence
ccaggagctgccaccgttcc

SEQ ID NO: 160 - DNA, 20 bp, Artificial sequence
  synthetic sequence
tgacttgctccctggaacgg

SEQ ID NO: 161 - DNA, 20 bp, Artificial sequence
  synthetic sequence
cctggaacggtggcagctcc

SEQ ID NO: 162 - DNA, 20 bp, Artificial sequence
  synthetic sequence
ctggaacggtggcagctcct

SEQ ID NO: 163 - DNA, 20 bp, Artificial sequence
  synthetic sequence
ggggccgggtctgcgttccc

SEQ ID NO: 164 - DNA, 20 bp, Artificial sequence
  synthetic sequence
agctcctgggaacgcagacc

SEQ ID NO: 165 - DNA, 20 bp, Artificial sequence
  synthetic sequence
ggcgcgggccagctggggcc

SEQ ID NO: 166 - DNA, 20 bp, Artificial sequence
  synthetic sequence
aggcgcgggccagctggggc

SEQ ID NO: 167 - DNA, 20 bp, Artificial sequence
  synthetic sequence
aacgcagacccggccccagc

SEQ ID NO: 168 - DNA, 20 bp, Artificial sequence
  synthetic sequence
aggaaggcgcgggccagctg

SEQ ID NO: 169 - DNA, 20 bp, Artificial sequence
  synthetic sequence
gaggaaggcgcgggccagct

SEQ ID NO: 170 - DNA, 20 bp, Artificial sequence
  synthetic sequence
cgaggaaggcgcgggccagc

SEQ ID NO: 171 - DNA, 20 bp, Artificial sequence
  synthetic sequence
cggggcctcgaggaaggcgc

SEQ ID NO: 172 - DNA, 20 bp, Artificial sequence
  synthetic sequence
ccggggcctcgaggaaggcg

SEQ ID NO: 173 - DNA, 20 bp, Artificial sequence
  synthetic sequence
gctggcccgcgccttcctcg

SEQ ID NO: 174 - DNA, 20 bp, Artificial sequence
  synthetic sequence
gctggccggggcctcgagga

SEQ ID NO: 175 - DNA, 20 bp, Artificial sequence
  synthetic sequence
cgctgctggccggggcctcg

SEQ ID NO: 176 - DNA, 20 bp, Artificial sequence
  synthetic sequence
ccgcgccttcctcgaggccc

SEQ ID NO: 177 - DNA, 20 bp, Artificial sequence
  synthetic sequence
gtcggtgtcgctgctggccg

SEQ ID NO: 178 - DNA, 20 bp, Artificial sequence
  synthetic sequence
ggtcggtgtcgctgctggcc

SEQ ID NO: 179 - DNA, 20 bp, Artificial sequence
  synthetic sequence
cggtcggtgtcgctgctggc

SEQ ID NO: 180 - DNA, 20 bp, Artificial sequence
  synthetic sequence
gcggcggtcggtgtcgctgc

SEQ ID NO: 181 - DNA, 20 bp, Artificial sequence
  synthetic sequence
gccagtagccagccccgtcc

SEQ ID NO: 182 - DNA, 19 bp, Artificial sequence
  synthetic sequence
gggtccaggccaagtaggt

SEQ ID NO: 183 - DNA, 23 bp, Artificial sequence
  synthetic sequence
tggctcttcacctttctagtccc

SEQ ID NO: 184 - DNA, 20 bp, Artificial sequence
  synthetic sequence
tcatggagcatgtactacaa

SEQ ID NO: 185 - DNA, 24 bp, Artificial sequence
  synthetic sequence
ggttatgccacagcttaatacaga

SEQ ID NO: 186 - DNA, 22 bp, Artificial sequence
  synthetic sequence
tgacacccctttaaaactttgg

SEQ ID NO: 187 - DNA, 20 bp, Artificial sequence
  synthetic sequence
gcccgtggggtccgatgctg

SEQ ID NO: 188 - DNA, 20 bp, Artificial sequence
  synthetic sequence
ggagctccatctgaatgagg

SEQ ID NO: 189 - DNA, 20 bp, Artificial sequence
  synthetic sequence
ggctggaattgtgtgacttg

SEQ ID NO: 190 - DNA, 20 bp, Artificial sequence
  synthetic sequence
gtatccgtggactcaccgtg

SEQ ID NO: 191 - DNA, 20 bp, Artificial sequence
  synthetic sequence
tacccatcccatcgaatgat

SEQ ID NO: 192 - DNA, 20 bp, Artificial sequence
  synthetic sequence
gcagcaactgcatcacaagt

(19) United States
(12) Patent Application Publication
(10) Pub. No.: US 2024/0067970 A1
(43) Pub. Date: Feb. 29, 2024

(54) Methods to Quantify Rate of Clonal Expansion and Methods for Treating Clonal Hematopoiesis and Hematologic Malignancies

(71) Applicants: The Board of Trustees of the Leland Stanford Junior University, Stanford, CA (US); The Regents of the University of Michigan, Ann Arbor, MI (US); Vanderbilt University, Nashville, TN (US)

(72) Inventors: Siddhartha Jaiswal, San Francisco, CA (US); Alexander Bick, Redwood City, CA (US); Joshua Weinstock, Redwood City, CA (US)

(21) Appl. No.: 18/271,417

(22) PCT Filed: Jan. 21, 2022
(86) PCT No.: PCT/US2022/013333

§ 371 Date: Jul. 7, 2023

Related U.S. Application Data
(60) Provisional application No. 63/141,333, filed on Jan. 25, 2021.
(60) Provisional application No. 63/274,331, filed on Nov. 1, 2021.

(51) Int. Cl. C12N 15/113; A61K 31/7088; A61K 38/46; C12Q 1/6883

(52) U.S. Cl. CPC C12N 15/1135; A61K 31/7088; A61K 38/465; C12Q 1/6883; C12N 2310/14; C12Q 2600/156

(57) ABSTRACT

Compositions and methods are provided for the analysis and treatment of conditions relating to clonal hematopoiesis of indeterminate potential (CHIP). In some embodiments, treatment is provided to reduce the progression of CHIP, particularly to reduce the progression to hematologic malignancy and/or heart disease. In other embodiments, methods are provided for determining clonal expansion, for example as a molecular diagnostic test that enables determination of clonal growth rate from a single sample. The method for determining clonal expansion can be applied to identify factors that influence clonal expansion, including environmental, metabolic, microbiome, and genetic.

### CROSS REFERENCE TO RELATED APPLICATION

The present application claims the benefit of and priority to U.S. Provisional Patent Application No. 63/141,333, filed Jan. 25, 2021, and U.S. Provisional Patent Application No. 63/274,331, filed Nov. 1, 2021 the entire disclosure of which is hereby.

### STATEMENT REGARDING FEDERALLY SPONSORED RESEARCH

This invention was made with Government support under contract HL15754001 and OD029586 awarded by the National Institutes of Health. The Government has certain rights in the invention.

### INCORPORATION BY REFERENCE OF SEQUENCE LISTING PROVIDED AS A TEXT FILE

A Sequence Listing is provided herewith in a text file, (S20-482_STAN-1811WO_SEQ_LIST_ST25.txt), created on Jan. 20, 2022, and having a size of 39,000 bytes. The contents of the text file are incorporated herein by reference in its entirety.

### BACKGROUND

Aging is characterized by the accumulation of somatic mutations, nearly all of which are “passengers” that have little fitness consequence on the cells in which they occur. However, infrequent fitness-increasing mutations, called “drivers”, may result in an expanded lineage of cells, termed a clone. Clonal hematopoiesis of indeterminate potential (CHIP) is defined by the acquisition of specific, cancer-associated driver mutations in hematopoietic stem cells (HSC) from persons without a blood cancer. Previous reports have associated CHIP with increased risk for hematologic malignancy, coronary heart disease, and mortality. The variant allele fraction (VAF), defined as the proportion of sequencing reads at a locus containing the mutant allele, is an approximate measure of clone size. In contrast to low VAF clones, which are ubiquitous in older individuals, large VAF CHIP clones are less common and more likely to result in hematologic malignancy and cardiovascular disease.

The genes commonly mutated in CHIP include regulators of DNA methylation (TET2, DNMT3A), chromatin remodeling (ASXL1), and RNA splicing (SF3B1, SRSF2, U2AF1). Even though these mutations are highly prevalent in CHIP and hematological cancers, the mechanisms driving clonal expansion remain largely unknown. This is partially due to a lack of sizable cohorts with serially sampled blood over decades which would otherwise enable studies on genetic and environmental correlates of clonal expansion.

The assessment and treatment of CHIP is of great clinical and research interest.

### SUMMARY

Compositions and methods are provided for the analysis and treatment of conditions relating to clonal hematopoiesis of indeterminate potential (CHIP). In some embodiments, treatment is provided to reduce the progression of CHIP, particularly to reduce the progression to hematologic malignancy and/or heart disease. In other embodiments, methods are provided for determining clonal expansion, for example in a method using a molecular diagnostic test that enables determination of clonal growth rate from a single sample. Methods for determining clonal expansion can be applied to identify factors that influence such clonal expansion, including environmental, metabolic, microbiome, and genetic factors. In some embodiments, a method is provided for diagnosing CHIP by determining the expression level of TCL1A, where increased expression of TCL1A is diagnostic for CHIP.

It is shown herein that increased expression of TCL1A is associated with increased clonal expansion. It is proposed that the TCL1A promoter is normally inaccessible and gene expression is low in hematopoietic stem cells. In the presence of driver mutations, e.g. and without limitation including driver mutations in one or more of TET2, ASXL1, SF3B1, SRSF2, JAK2, etc., the TCL1A promoter opens, permitting gene expression and driving clonal expansion of the mutated cells. The presence of the alt-allele at human SNP rs2887399 prevents accessibility of chromatin at the TCL1A promoter, leading to reduced expression of TCL1A RNA, and abrogated clonal advantage due to the mutations. It was found that down-regulating TCL1A can prevent clonal expansion.

In some embodiments, an individual identified as having CHIP is treated with an agent to reduce TCL1A expression or activity. In some embodiments, hematopoietic stem cells of the individual are engineered to have reduced expression of TCL1A, e.g. by in vitro modification of the promoter of coding sequence of TCL1A to reduce expression; using CRISPR induced frameshifts to prevent the development of leukemia in those undergoing HSCT, e.g. during genetic correction of autologous hematopoietic stem cells (HSC) in sickle-cell disease; and the like. In some embodiments the individual is treated with an agent that reduces TCL1A expression, e.g. in circulating cells, in bone marrow, etc. Such an agent includes, without limitation, anti-sense oligonucleotides specific for TCL1A, RNAi agents specific for TCL1A, small molecule inhibitors of TCL1A activity, antibodies and antibody fragments specific for the inhibition of TCL1A, and the like. The treatment may be combined with administration of additional agents or regimens useful in the treatment of hematologic malignancies. The treatment can provide for a reduction in the development of hematologic cancers, including without limitation acute myeloid leukemia, myelodysplastic syndrome, myeloproliferative neoplasms, chronic myeloid leukemia, chronic myelomonocytic leukemia, and diffuse large B-cell lymphoma, as well as heart disease and death in persons with clonal hematopoiesis, who are at risk for these conditions.

In some embodiments, an individual selected for CHIP treatment is genotyped for SNP rs2887399 prior to treatment, and found to have the reference allele. In some embodiments an individual selected for CHIP treatment described herein is genotyped for the presence of a driver mutation in one or more of TET2, ASXL1, SF3B1, SRSF2, TP53, JAK2, PPM1 D, NRAS, KRAS, IDH1, and IDH2 prior to treatment, and found to have at least one such driver mutation.

In some embodiments a method is provided for diagnosing or predicting clonal hematopoiesis of indeterminate potential (CHIP) in an individual, the method comprising: detecting in the individual a genetic mutation that increases TCL1 activity; or determining increased expression of TCL1A.

In some embodiments, methods are provided for screening a candidate agent for treatment of CHIP, the methods comprising selecting an agent that down-regulates expression of TCL1A or reduces activity of TCL1A, and determining the effect of the agent on clonal expansion of hematopoietic cells.

In other embodiments, methods are provided for determining the clonal growth rate of a hematopoietic clone from a sample, e.g. a peripheral blood sample, using PACER (passenger-approximated clonal expansion rate). Passenger counts represent a composite measure of the fitness and birth date of an underlying clone and provides a simple predictor of clonal expansion. In some embodiments the determination is performed on a single sample, i.e. in the absence of a time course of samples. In some embodiments an individual is treated in accordance with the findings of the clonal growth determination, where treatment may comprise administration of an agent or regimen that reduces the number of cells in a clone.

The inventive methods of determining clonal growth are based on sequence analysis of mutations present in the clone. While a clone, e.g. a clone of hematopoietic stem cells, accumulates mutations, most are passenger mutations that do not have any significant consequence on the stem cells ability to divide or proliferate. These passenger mutations are largely undetectable until the stem cell acquires a somatic mutation in a driver gene that provides the clone with a clonal advantage, e.g. mutations in one or more of DNMT3A, TET2, ASXL1, JAK2, etc. DNA sequencing a peripheral blood sample from an individual with CHIP identifies CHIP driver mutations, and also a body of passenger mutations. The number of passenger mutations (passenger counts) is used to estimate clone age. As clonal hematopoietic blood clones expand, the variant allele fraction of both driver and passenger mutations increases. It is shown that the passenger mutations are likely to precede the driver mutation. As the passenger mutations accrue at a constant rate across time that is similar across individuals, they can be used to date the acquisition of the driver. For example, in two individuals of the same age and with clones of the same size, the clone with more passenger mutations has greater growth potential, as it expanded to the same size in less time. Higher growth potential clones will harbor more detectable passengers than lower fitness clones that arose at the same time.

In some embodiments, the presence of passenger mutations in a hematopoietic sample from an individual suspected of having CHIP provides a composite measure of clone fitness and clone birth date, using the PACER method described above.

In some embodiments, genetic sequencing of a hematopoietic sample first identifies non-reference variants in the genomes using standard algorithms, selecting for variants that are present at variant allele frequencies below the threshold for a germline variant. To reduce the likelihood of recurrent sequencing artifacts, somatic variants that were found only in a single individual in a dataset may be used. As different mutation sub-types vary in their association with age at blood draw, only C-T and T-C mutations may be selected, as these are the most strongly age-associated. These steps provide identification of a set of variants in the genomes referred to as passengers. In some embodiments the steps are embodied as a program of instructions executable by computer and performed by means of software components loaded into the computer. The passenger count is then used to determine clone fitness and clone birth date. In some embodiments, the passenger count is compared to a reference sample, e.g. an individual with a known CHIP clone date and/or size.

### BRIEF DESCRIPTION OF THE DRAWINGS

FIG. 1. PACER Enables Estimation of Clonal Expansion from a Single Blood Draw. A, A schematic depiction of using passenger counts to estimate the rate of expansion of a hematopoietic stem cell (HSC) clone after the acquisition of a driver mutation. The passengers (blue) that precede the driver (red) can be used to date the acquisition of the driver. B, The observed clonal expansion rates (dVAFdT), as expressed in the change in variant allele frequency (VAF) over time (years), were associated with increased passenger counts in 55 CHIP carriers from the Women's Health Initiative. Colors indicate the mutated driver gene. C, A multivariate model including passenger counts, age at blood draw, and VAF indicates the relative contributions of age and VAF over baseline models. AIC is Akaike information criteria, where smaller values indicate better model fit. D, The relative abundances of passenger counts were estimated for CHIP driver genes with at least 30 cases using a negative binomial regression, adjusting for age at blood draw, driver VAF, and study. The coefficients are relative to DNMT3A R882− CHIP.

FIG. 2. GWAS of PACER Identifies Germline Determinants of Clonal Expansion in Blood. A, A genome-wide association study (GWAS) of passenger counts identifies TCL1A as a genome-wide significant locus. B, The association between the genotypes of rs2887399 and PACER varied between TET2 and DNMT3A. Alt-alleles were associated with decreased PACER score in TET2 mutation carriers, in contrast to DNMT3A carriers, where no association was observed. C, The association between alt-alleles at rs2887399 and presence of specific CHIP mutations varies by CHIP mutations. Forest plot shows the effect estimates of a single T allele and two T-alleles respectively, estimating using Firth logistic regression. On the right of the forest plot, effect estimates and p-values are included from SAIGE 23, which uses an additive coding of the alt-alleles for hypothesis testing. In the additive tests, SF3B1 and SRSF2 were grouped together to aid convergence.

FIG. 3. TET2 and ASXL1 mutations permit aberrant TCL1A accessibility and transcript expression in HSCs and MPPs. A. Quantification of fraction of HSCs and MPPs expressing TCL1A transcripts in patients with TET2 or ASXL1 driven acute myeloid leukemia (AML) or myeloproliferative neoplasm (MPN) compared to healthy donors. Data is from single-cell RNA sequencing generated in Psaila et al ³⁶ and Velten et al ³⁶. B. ATAC-sequencing tracks of the TCL1A locus near rs2887399 in HSCs form healthy donors (row 1), pre-leukemic hematopoietic stem cells (pHSCs) from patients with AML but no detected driver mutations (rows 2-3), pHSCs with DNMT3A mutations (rows 4-5), and in pHSCs with TET2 mutations (rows 6-7). Amino acid change and variant allele fraction (VAF) for the driver mutations are shown. Data is from Corces et al⁶⁵. Vertical grey bar indicates location of the rs2887399 SNP. Black hash marks indicate positions of GTEX v8 eQTLs for TCL1A in whole blood, blue hash marks indicate positions of genome-wide significant SNPs, and the red hash mark indicates the position of the single causal variant identified by fine-mapping, rs2887399.

FIG. 4. T allele of rs2887399 reduces TCL1A expression and extinguishes clonal expansion phenotype of TET2 and ASXL1 mutant HSPCs. A. Schematic of experimental workflow. Human HSPCs from donors carrying rs2887399 GG, GT, or TT genotypes were electroporated with Cas9 targeting AAVS1, TET2, DNMT3A, or ASXL1 and cultured for OMNI-ATAC, intracellular flow cytometric analysis of TCL1A expression, or an in vitro HSPC expansion assay. B. ATAC-sequencing tracks illustrating chromatin accessibility at rs2887399 in TET2-edited HSPCs cultured for 5 days from donors of the GG, GT, and TT genotypes. Red line indicates location of rs2887399. C. Representative intracellular flow plots of TCL1A protein expression in edited HSCs/MPPs from each rs2887399 donor after 11 days in culture. D. Quantification of percent HSCs/MPPs expressing TCL1A from flow cytometry, stratified by edited gene and rs2887399 genotype. Results of a linear regression model for the effect of edited gene (referent to AAVS1), number of T-alleles at rs2887399, and the interaction term of edited gene with T-alleles are presented below. Est.=estimate, S.E.=standard error, p. val.=p-value. E. Quantification of Lin- CD34+ CD38− CD45RA− HSC/MPP counts after 14 days of in vitro expansion stratified by edited gene and rs2887399 genotype. Results of a linear regression model for the effect of edited gene (referent to AAVS1), rs2887399 genotype (referent to GG), and the interaction term of edited gene with rs2887399 genotype are presented below. F. Quantification of Lin-/lo CD34+ CD38− CD45RAlo HSPCs (CD45RAlo HSPCs) after 14 days of in vitro expansion stratified by edited gene and rs2887399 genotype. Results of a linear regression model for the effect of edited gene (referent to AAVS1), rs2887399 genotype (referent to GG), and the interaction term of edited gene with rs2887399 genotype are presented below.

FIG. 5. CHIP Carriers are Enriched for Passengers. The passenger counts are enriched by 54% (95% CI: 51%-57%) after adjusting for age and study using a negative binomial regression. The different colors in the density plots correspond to quartiles of the marginal probability distributions. As the density estimates are smoothed, the underlying data points are indicated with hash marks. The data use a log2 scale, such that an increase by 1 indicates a single doubling has occurred.

FIG. 6. Passenger Counts Linearly Increase with Number of Driver Mutations. The distributions of passenger counts are stratified by the number of CHIP driver variants acquired. The different colors in the density plots correspond to quartiles of the marginal probability distributions.

FIG. 7. Fine-mapping TCL1A Locus Identifies a Single Causal Variant rs2887399. The posterior inclusion probabilities (PIP) as estimated by SuSIE are plotted on the y-axis, and the genomic position of a 0.8 Mb region including TCL1A is plotted on the x-axis. The linkage disequilibrium (LD) estimates are plotted on a color scale and are estimated on the genotypes used for association analyses.

FIG. 8. Rare Variant Analysis Of TCL1A Locus Identifies a Suggestive Signal Prior to Conditioning on rs2887399. Rare variant analyses were performed using the SCANG⁵⁶ rare variant scan procedure including all variants with a minor allele count less than 300. Identified rare variant windows are plotted as gray rectangles where the width corresponds to the size of the genomic region and the height corresponds to the pvalue of the SCANG test statistic for the window.

FIG. 9. Conditioning on rs2887399 Attenuates Independent Rare Variant Signal. Rare variant analyses were performed including the rs2887399 genotypes as covariate.

FIG. 10. TCL1A Promoter is Not Well Conserved In Vertebrates. Multiz alignments across multiple species are shown for the TCL1A locus.

FIG. 11. PACER Signal Colocalizes with TCL1A eQTLs. In the top panel, plotted are the −log10 pvalues from both the PACER GWAS and TCL1A cis-eQTLs in whole blood from GTEx v8. In the bottom panel, posterior probability of colocalization from COLOC identifies rs2887399 as the likely shared causal variant.

FIG. 12. Schematic Description of rs2887399 Mediation on TET2 Clonal Expansion. Proposed model for clonal advantage due to mutations in TET2. In cells with the rs2887399 REF/REF genotype, loss of TET2 function leads to an accessible TCL1A locus, aberrant TCL1A RNA and protein expression in hematopoietic stem cells (HSC's) and multi-potent progenitors (MPP's), and subsequent clonal expansion. The presence of rs2887399 ALT alleles diminishes the TET2 clonal expansion phenotype by limiting TCL1A locus accessibility and downstream protein expression.

FIG. 13. CRISPR Editing Efficiency. A. ICE analysis of Sanger traces to determine targeted CRISPR editing efficiency. Bar plots display percent of CD34+ CD38− CD45RA− cells with indel formation in gene of interest. These cells were used for the OMNI-ATAC and intracellular TCL1A flow assays. B. ICE analysis of Sanger traces to determine targeted CRISPR editing efficiency. Bar plots display percent of CD34+ CD38− CD45RA− cells with indel formation in gene of interest. These cells were used for the 14 day expansion assay.

FIG. 14. HSC/MPP Flow Gating Scheme. Flow gating scheme for identifying and sorting CD34+ CD38− CD45RA− hematopoietic stem cells (HSC's) and multi-potent progenitors (MPP's). The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Clonal hematopoiesis of indeterminate potential (CHIP). CHIP defines patients who have detectable somatic clonal mutations in genes recurrently mutated in hematologic malignancies, but who lack a known hematologic malignancy or other clonal disorder. Under this definition, CHIP encompasses cytopenic patients with concurrent cancer-associated mutations who do not meet diagnostic criteria for MDS, as well as those with normal peripheral blood counts. CHIP would not include clearly described clonal conditions such as paroxysmal nocturnal hemoglobinuria, MBL, or MGUS. In general as a working definition, the mutant allele fraction must be ≥2% in the peripheral blood, because with deep enough sequencing, a mutation can be found in every individual, and current outcomes data are based on a minimum variant allele fraction of >2% in peripheral blood. Variants present below this threshold are not known to carry increased risk of adverse outcomes. A copy number variant resulting from a chromosomal rearrangement involving a chromosomal region where hematologic neoplasia-associated genes are encoded is also consistent with CHIP.

CHIP is distinct from MDS because CHIP is associated with a much longer survival, normal blood counts in most cases, and low rate of progression to AML. Individuals with CHIP have an increased risk of disease progression to hematologic neoplasia compared with individuals without detectable mutations, and this risk appears to be proportional to the size of the somatic clone; however, the rate of progression appears to be only 0.5% to 1% per year, similar to MBL and MGUS. Although the annual rate of progression of CHIP, MBL, and MGUS to overt neoplasia is comparable, MBL and MGUS represent expansions of lineage-committed cells, whereas CHIP involves hematopoietic stem cells or less mature progenitor cells, and thus CHIP is a precursor state for a broader range of hematologic neoplasms.

Minimal diagnostic criteria for MDS requires the presence of blood cytopenias and exclusion of reactive or other nonhematopoietic causes of those cytopenias. In addition, of the following diagnostic features must be present to diagnose MDS: excess blasts (≥5%) with a myeloid phenotype (but <20% blasts, which would qualify as AML); >10% dysplastic cells in at ≥1 of the 3 myeloid lineages (erythroid, granulocytic, megakaryocytic) or ≥15% ring sideroblasts as a proportion of erythroid precursors; or evidence of clonality as manifested by an abnormal MDS-associated karyotype. If the latter group of cytogenetically abnormal cases does not meet blast or dysplasia criteria for MDS diagnosis, they are diagnosed as “MDS, unclassifiable” and have a natural history similar to MDS. Co-criteria for diagnosis that might be useful in difficult cases, such as decreased circulating colony-forming cells, abnormal flow cytometric immunophenotype, aberrant gene expression pattern, or the presence of an MDS-associated somatic mutation.

The terms “cancer”, “neoplasm”, “tumor”, and “carcinoma”, are used interchangeably herein to refer to cells that exhibit relatively autonomous growth, so that they exhibit an aberrant growth phenotype characterized by a significant loss of control of cell proliferation. In general, cells of interest for detection or treatment in the present application include without limitation precancerous, malignant, pre-metastatic, metastatic, and non-metastatic cells. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc. As disclosed above, CHIP includes cells that have expanded but do not yet have a malignant phenotype.

The terms “hematological malignancy”, “hematological tumor”, and “hematological cancer” are used interchangeably and in the broadest sense herein and refer to all stages and all forms of cancer arising from cells of the hematopoietic system.

Examples of hematologic malignancies include leukemias, lymphomas, and myelomas, including but not limited to acute biphenotypic leukemia, acute myelogenous leukemia (AML), acute lymphoblastic leukemia (ALL), acute promyelocytic leukemia (APL), biphenotypic acute leukemia (BAL) blastic plasmacytoid dendritic cell neoplasm, chronic myelogenous leukemia (CML), chronic myelomonocytic leukemia (CMML), chronic lymphocytic leukemia (CLL) (called small lymphocytic lymphoma (SLL) when leukemic cells are absent), acute monocytic leukemia (AMOL), Hodgkin's lymphomas, Non-Hodgkin's lymphomas (e.g. chronic lymphocytic leukemia (CLL), diffuse large B-cell lymphoma (DLBCL), Follicular lymphoma (FL), Mantle cell lymphoma (MCL), Marginal zone lymphoma (MZL), Burkitt's lymphoma (BL), Hairy cell leukemia, Post-transplant lymphoproliferative disorder (PTLD), Waldenstrom's macroglobulinemia/lymphoplasmacytic lymphoma, hepatosplenic-T cell lymphoma, and cutaneous T cell lymphoma (including Sezary's syndrome)), multiple myeloma, myelodysplastic syndrome, and myeloproliferative neoplasms. In particular embodiments, the subject methods find utility in addressing the development of hematologic malignancies associated with CHIP, e.g. acute myeloid leukemia, myelodysplastic syndrome, myeloproliferative neoplasms, chronic myeloid leukemia, chronic myelomonocytic leukemia, and diffuse large B-cell lymphoma.

Variant allele fraction (VAF). The proportion of genomes in a population, which may be defined as sequencing reads, that contain a mutant allele at a locus of interest. This provides an approximate measure of clone size.

Clonal growth rate. As used herein, the term clonal growth rate refers to an empirical measurement of how clone size, which can be expressed as VAF, changes over time.

Clonal fitness. As used herein, the term clonal fitness is defined as the proliferative advantage of a cells carrying a mutation over cells carrying no or only neutral mutations. It may be expressed as the percent increase in growth that exceeds normal cell growth.

Birth date. As used herein the term refrs to the time at which a mutation arose.

Passenger mutation, as used herein, refers to a somatic mutation in a cell that does not alter clonal fitness, but occurs in a cell that coincidentally or subsequently acquires a driver mutation.

Driver mutation, as used herein, refers to a somatic mutation in a cell that confers a selective growth advantage to the cell, i.e. it increases clonal fitness.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with respect to a patient encompasses bone marrow, e.g. bone marrow aspirate; blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample comprising target cells or normal control cells or suspected of comprising such cells or biological fluids derived therefrom (e.g., cancerous cell, etc.), e.g., a sample comprising polynucleotides and/or polypeptides that is obtained from such cells (e.g., a cell lysate or other cell extract comprising polynucleotides and/or polypeptides). A biological sample comprising tumor cells from a patient can also include non-tumor cells.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc.

Circulating and bone marrow blast cells. It is typical of leukemias and myelodysplastic syndromes that tumor cells are found in the circulation and bone marrow. The number of blast cells, or white blood cells can be counted in these tissues. Counting blast cells can be more accurate, as the percentage of WBC that are blasts can vary with the condition.

Cells for use in the methods as described herein may be collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxic cells, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; (b) relieving the disease or its symptoms, i.e., causing regression of the disease or its symptoms; and (c) preventing progression to a disease state.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to prevent, delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

TCL1A consists of 114 amino acids, has a predicted molecular weight of 14 kDa, and the protein has a unique symmetrical β-barrel structure. In the lymphoid compartment, TCL1A expression is limited to CD4⁻CD8⁻CD3⁻ thymocytes as well as CD34⁺CD19⁺ pro-B cell through IgM-negative pre-B cells. TCL1 is an Akt kinase coactivator, which facilitates the oligomerization and activation of Akt in vivo. Consequently, it promotes Akt-dependent cell survival. Reference sequences for human TCL1A include Genbank mRNA NM_001098725 and NM_021966; protein NP_001092195 and NP_068801.

The TCL1 gene family, consisting of TCLl a (also called TCL1), TCL1b(also called TML1), MTCP1, TNG1 and TNG2 isoforms in human, are a group of proto-oncogenes whose proteins were initially identified in the translocation of human T-PLL. Under physiological conditions,TCL1 transcripts are preferentially expressed in cells of lymphoid lineages and mainly in immature CD4⁻CD8⁻ cells during development, but not in either CD4⁺ or CD8⁺ mature T cells in circulation. Studies have demonstrated the role of TCL1a as an Akt kinase co-activator that promotes kinase activity and transphosphorylation of Akt, thus promoting its nuclear transport. Activation of Akt leads to cell survival, which underlies the pathogenic mechanism of numerous neoplastic diseases such as lung, ovarian and prostate cancer. Therefore, over-expression of TCLl a could modulate and amplify Akt activation, allowing enhanced signal transduction, cell proliferation and survival, which forms the basis of malignancies.

The proteins encoded by genes in the TCL1 family are conserved between members, whereas none of them are matched with any known proteins, therefore they are characterized into a novel family of proteins. The structure of TCL1 a protein is a β barrel with an internal hydrophobic core, which consists of two four-stranded β sheets connected by a long loop. Strands βA, βB, βE, and βF are 4 long boards forming one side of the barrel, while the other side of the barrel is composed of 4 short strands βC, βD, βG and βH. Approximately 40% homology has been found between the TCLl a and TCL1b protein, including most amino acids which forms the hydrophobic core. The A1 transcript is a small cysteine-rich coiled-coil protein composed of three a helices, among which two antiparallel helices form an a hairpin stabilized by two disulfide bridges and inter-helix hydrophobic contacts.

TCL1 proteins act as co-activators to influence the signaling transduction of Akt that might play a role in promoting cell survival, proliferation, growth and metabolism. In the Akt pathway, signal transduction is initiated by the activation of phosphatidylinositol 3-kinase (PI3K) via tyrosine kinase receptors. Activated PI3K forms phosphatidylinositol-3,4-biphosphate (PIP2) and phosphatidylinositol-3,4,5-triphosphate(PIP3) in the plasma membrane, which is tightly regulated by phosphatases. The combination of the pleckstrin homology (PH) domain of Akt with the inositol head group of PIP3 recruits Akt to the plasma membrane with conformational conversion. After being phosphorylated at the site of Thr-308 and Ser-473 by 3-phosphatidyinositol-dependentkinase 1 (PDK1) and another kinase, Akt is disassociated from the membrane into the cytosol to phosphorylate downstream proteins.

TCL1 proteins including TCL1a, TCL1b and MTCP1 can bind to Akt and appear to have effects on promoting Akt kinase activation and nuclear translocation by interacting with Akt. For TCL1a, co-immunoprecipitation experiments have shown that the interaction of TCL1a with Akt facilitates Akt conformational exchange. TCL1 a may induce Akt phosphorylation at the site of Ser-473 and Thr-308 and enhance Akt activity though synergic effects instead of activating the Akt kinase directly. The structures of TCL1a and Akt suggest their interaction pattern. Akt kinase contains a polarized PH domain, which is critical for Akt activation by binding with PIP3.0ne terminal of the PH domain is capped by a C-terminal amphipathica-helix with two antiparallel β sheets, while the other terminal is formed by three variable loops, VL1, VL2 and VL3, as the phospholipid-binding site. The β5 and β6 strand and the α-helix at the PH domain form a site where could be combined with the exposed 2AA hydrophobic patch at one terminal of the β barrel of TCL1a. Since a dimeric structure is required for TCL1a to have biological functions, two TCL1a-bound Akt kinases are then cross-linked with intactness of other PH-ligand interactions to form a TCL1a-Akt homodimer complex, which ultimately strengthens membrane association, promotes Akt phosphorylation and inhibits Akt inactivation. Therefore, by increasing the Akt-mediated phosphorylation of downstream substrates, such as BAD and GSK-3, TCL1a is able to promote cell proliferation, stabilize mitochondrial transmembrane potential and promote cell survival.

Furthermore, the interaction between TCL1a and Akt may also contributes to Akt nuclear translocation. Akt is mainly expressed in the cytoplasm, while TCL1a is distributed in both the cytoplasm and the nucleus. Immunofluorescence assays have indicated that Akt and TCL1a are co-localized in the cytoplasm and the nucleus in cells with co-expression ofTCL1a and Akt, meanwhile the TCL1a-Akt interaction in the cytoplasm contributes to the nuclear translocation of Akt.

SNP rs2887399 (at human genome position chr14:95714358 (GRCh38.p13)) is of interest for genotyping TCL1A. The reference allele of the SNP has forward strand G at the site of polymorphism, while the alt allele has T. It is shown herein that the alt allele can be protective of progression to malignancy from CHIP. Sequence analysis can be used to detect specific polymorphisms in a nucleic acid, for example where a test sample of DNA or RNA is obtained from the test individual. PCR or other appropriate methods can be used to amplify the gene or nucleic acid, and/or its flanking sequences, if desired. The sequence of an SNP in the nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA or mRNA, as appropriate. Allele-specific oligonucleotides can also be used to detect the presence of a polymorphism in a nucleic acid, through the use of amplification, dot-blot hybridization of amplified oligonucleotides with allele-specific oligonucleotide (ASO) probes, etc.

Another SNP, 10 base pairs away from rs2887399, can also be used for genotyping (rs11846938). The REF allele for rs11846938 is a T, the ALT allele is G. The two SNPs are strongly in linkage disequilibrium.

An anti-TCL1A agent is defined as an agent that selectively reduces activity of TCL1A in a targeted cell, for example with a targeted small molecule, antibody or antibody fragment, gene editing system, siRNA, shRNA, and the like. Examples include those set forth in Table 1 and Table 2.

TABLE 1
shRNA sequences

| | SEQ ID | |
|---|---|---|
| Gene | NO: | Forward Oligo Sequence |
| TCL1A | SEQ ID | CCGGCTGGGAGAAGTTCGTGTATTTCTCGAGAAA |
| | NO: 1 | TACACGAACTTCTCCCAGTTTTTG |
| TCL1A | SEQ ID | CCGGGCCCTTAACCATCGAGATAAACTCGAGTTT |
| | NO: 2 | ATCTCGATGGTTAAGGGCTTTTTG |
| TCL1A | SEQ ID | CCGGTGCCCTTAACCATCGAGATAACTCGAGTTA |
| | NO: 3 | TCTCGATGGTTAAGGGCATTTTTG |
| TCL1A | SEQ ID | CCGGGCATGTAAACACGCCTGCAAACTCGAGTTT |
| | NO: 4 | GCAGGCGTGTTTACATGCTTTTTG |
| TCL1A | SEQ ID | CCGGGCGCTTAGTGTACCACATCAACTCGAGTTG |
| | NO: 5 | ATGTGGTACACTAAGCGCTTTTTG |
| TCL1A | SEQ ID | TACTATGCCTGTGTCTTCTCCACCACGCTTCAAG |
| | NO: 6 | AGAGCGTGGTGGAGAAGACACAGGCATAGTA |
| TCL1A | SEQ ID | CTGGATGATGGTCTTCAGCCTCTTTCTGTTCAAG |
| | NO: 7 | AGACAGAAAGAGGCTGAAGACCATCATCCAG |
| TCL1A | SEQ ID | GGAGGAATGGACAGACAGAGGATGAGCTCTCAAG |
| | NO: 8 | CTAGGAGCTCATCCTCTGTGTCCATTCCTCC |
| TCL1A | SEQ ID | TCCATTTGGCAGAGCTTCTTCCAGGTGCCTCAAG |
| | NO: 9 | AGGGCACCTGGAAGAAGCTCTGCCAAATGGA |
| TCL1A | SEQ ID | agatctGGTAATGATTGATTAAATAtacctgacc |
| | NO: 10 | cataTATTTAATCAATCATTACCTTTTTggtacc |
| TCL1A | SEQ ID | agatctGTATTCATGGATTTATTTAtacctgacc |
| | NO: 11 | cataTAAATAAATCCATGAATACTTTTTggtacc |
| TCL1A | SEQ ID | agatctGATATTCATGGATTTATTAtacctgacc |
| | NO: 12 | cataTAATAAATCCATGAATATCTTTTTggtacc |
| TCL1A | SEQ ID | agatctGCATGGATTTATTTATTTAtacctgacc |
| | NO: 13 | cataTAAATAAATAAATCCATGCTTTTTggtacc |
| TCL1A | SEQ ID | agatctGATTCATGGATTTATTTAAtacctgacc |
| | NO: 14 | cataTTAAATAAATCCATGAATCTTTTTggtacc |
| TCL1A | SEQ ID | agatctGAATATTCATGGATTTATAtacctgacc |
| | NO: 15 | cataTATAAATCCATGAATATTCTTTTTggtacc |
| TCL1A | SEQ ID | agatctGTTGTAATGATTGATTAAAtacctgacc |
| | NO: 16 | cataTTTAATCAATCATTACAACTTTTTggtacc |
| TCL1A | SEQ ID | agatctGCAAGATAGTTTATCTAAAtacctgacc |
| | NO: 17 | cataTTTAGATAAACTATCTTGCTTTTTggtacc |
| TCL1A | SEQ ID | agatctGGAGAAGTTCGTGTATTTAtacctgacc |
| | NO: 18 | cataTAAATACACGAACTTCTCCTTTTTggtacc |
| TCL1A | SEQ ID | agatctGTAATGATTGATTAAATGAtacctgacc |
| | NO: 19 | cataTCATTTAATCAATCATTACTTTTTggtacc |
| TCL1A | SEQ ID | agatctGTTTGTAATGATTGATTAAtacctgacc |
| | NO: 20 | cataTTAATCAATCATTACAAACTTTTTggtacc |
| TCL1A | SEQ ID | agatctGTTCATGGATTTATTTATAtacctgacc |
| | NO: 21 | cataTATAAATAAATCCATGAACTTTTTggtacc |
| TCL1A | SEQ ID | agatctGGATTAAATGCAAGATAGAtacctgacc |
| | NO: 22 | cataTCTATCTTGCATTTAATCCTTTTTggtacc |
| TCL1A | SEQ ID | agatctGTCATGGATTTATTTATTAtacctgacc |
| | NO: 23 | cataTAATAAATAAATCCATGACTTTTTggtacc |
| TCL1A | SEQ ID | agatctGAAATATTCATGGATTTAAtacctgacc |
| | NO: 24 | TcataTTAAATCCATGAATATTTCTTTTggtacc |
| TCL1A | SEQ ID | agatctGATTAAATGCAAGATAGTAtacctgacc |
| | NO: 25 | cataTACTATCTTGCATTTAATCTTTTTggtacc |
| TCL1A | SEQ ID | agatctGTGTATGTGGCATGAATAAtacctgacc |
| | NO: 26 | cataTTATTCATGCCACATACACTTTTTggtacc |
| TCL1A | SEQ ID | agatctGGCAAGATAGTTTATCTAAtacctgacc |
| | NO: 27 | cataTTAGATAAACTATCTTGCCTTTTTggtacc |
| TCL1A | SEQ ID | agatctGTAAATGCAAGATAGTTTAtacctgacc |
| | NO: 28 | cataTAAACTATCTTGCATTTACTTTTTggtacc |
| TCL1A | SEQ ID | agatctGAATGCAAGATAGTTTATAtacctgacc |
| | NO: 29 | cataTATAAACTATCTTGCATTCTTTTTggtacc |

TABLE 2
guide RNA sequences

| Target | | sgRNA Sequence |
|---|---|---|
| TCL1A CDS | SEQ ID NO: 31 | CGAGTGCCCGACACTCGGGG |
| TCL1A CDS | SEQ ID NO: 32 | TGTAACCTATCCTTTATCTG |
| TCL1A CDS | SEQ ID NO: 33 | TGCCTCTCAGATTGACGGCG |
| TCL1A CDS | SEQ ID NO: 34 | AGTTACGGGTGCTCTTGCGT |
| TCL1A CDS | SEQ ID NO: 35 | GCGCTTAGTGTACCACATCA |
| TCL1A CDS | SEQ ID NO: 36 | AGGATCGGTATCGTCCATCA |
| TCL1A CDS | SEQ ID NO: 37 | CTGGCTGCCCTTAACCATCG |
| TCL1A CDS | SEQ ID NO: 38 | ATGGCCGAGTGCCCGACACT |
| TCL1A CDS | SEQ ID NO: 39 | GGCCGAGTGCCCGACACTCG |
| TCL1A CDS | SEQ ID NO: 40 | GGGTAGAGCTGCCACATGAT |
| TCL1A CDS | SEQ ID NO: 41 | CAAGCCTGCTGCCTATCATG |
| TCL1A CDS | SEQ ID NO: 42 | GTTACGGGTGCTCTTGCGTC |
| TCL1A CDS | SEQ ID NO: 43 | GTCGGGAAGACGTCGTCCTG |
| TCL1A CDS | SEQ ID NO: 44 | CTCTCAGATTGACGGCGTGG |
| TCL1A CDS | SEQ ID NO: 45 | GGGAAGACGTCGTCCTGGGG |
| TCL1A CDS | SEQ ID NO: 46 | GAGGATCGGTATCGTCCATC |
| TCL1A CDS | SEQ ID NO: 47 | CAAATACACGAACTTCTCCC |
| TCL1A CDS | SEQ ID NO: 48 | AAAGGATAGGTTACAGTTAC |
| TCL1A CDS | SEQ ID NO: 49 | TGGCTGTACCTCGATGGTTA |
| TCL1A CDS | SEQ ID NO: 50 | CGTCGGGAAGACGTCGTCCT |
| TCL1A CDS | SEQ ID NO: 51 | AGAAACTGGAGTCTGAGGAT |
| TCL1A CDS | SEQ ID NO: 52 | GCTGCCACATGATAGGCAGC |
| TCL1A CDS | SEQ ID NO: 53 | CAAAGCTGGCTGTACCTCGA |
| TCL1A CDS | SEQ ID NO: 54 | GAGTGTCGGGCACTCGGCCA |
| TCL1A CDS | SEQ ID NO: 55 | GGCTGTACCTCGATGGTTAA |
| TCL1A CDS | SEQ ID NO: 56 | CTCACCATACATCAGTCATC |
| TCL1A CDS | SEQ ID NO: 57 | GCGTCGGGAAGACGTCGTCC |
| TCL1A CDS | SEQ ID NO: 58 | GGAGGCAGTCACCGACCACC |
| TCL1A CDS | SEQ ID NO: 59 | GAAAGACACTCACCTTGATG |
| TCL1A CDS | SEQ ID NO: 60 | GTACACTAAGCGCCAGAAAC |
| TCL1A CDS | SEQ ID NO: 61 | ACTTCTCCCAGGCCCACAGG |
| TCL1A CDS | SEQ ID NO: 62 | GTGACTGCCTCCCCGAGTGT |
| TCL1A CDS | SEQ ID NO: 63 | CTCCCCGAGTGTCGGGCACT |
| TCL1A CDS | SEQ ID NO: 64 | GCGCCAGAAACTGGAGTCTG |
| TCL1A CDS | SEQ ID NO: 65 | TGGACGAGAAGCAGCACGCC |
| TCL1A CDS | SEQ ID NO: 66 | AGGCCCACAGGCGGTCCGGG |
| TCL1A CDS | SEQ ID NO: 67 | ATGTGGCAGCTCTACCCTGA |
| TCL1A CDS | SEQ ID NO: 68 | AGGCTTGGGCCTATCTGGGT |
| TCL1A CDS | SEQ ID NO: 69 | TGGCCGAGTGCCCGACACTC |
| TCL1A CDS | SEQ ID NO: 70 | ACATGATAGGCAGCAGGCTT |
| TCL1A CDS | SEQ ID NO: 71 | CCGACCACCCGGACCGCCTG |
| TCL1A CDS | SEQ ID NO: 72 | CCTATGACCCCCACCCAGAT |
| TCL1A CDS | SEQ ID NO: 73 | TGACTGCCTCCCCGAGTGTC |
| TCL1A CDS | SEQ ID NO: 74 | CGACCACCCGGACCGCCTGT |
| TCL1A CDS | SEQ ID NO: 75 | CTGGGAGAAGTTCGTGTATT |
| TCL1A CDS | SEQ ID NO: 76 | TGGGGGTCATAGGCCTCCCC |
| TCL1A CDS | SEQ ID NO: 77 | CGAACTTCTCCCAGGCCCAC |
| TCL1A CDS | SEQ ID NO: 78 | TAAAGGATAGGTTACAGTTA |
| TCL1A CDS | SEQ ID NO: 79 | CCACAGGCGGTCCGGGTGGT |
| TCL1A CDS | SEQ ID NO: 80 | TCCCAGGCCCACAGGCGGTC |
| TCL1A CDS | SEQ ID NO: 81 | CTCGATGGTTAAGGGCAGCC |
| TCL1A CDS | SEQ ID NO: 82 | CAGCAGGCTTGGGCCTATCT |
| TCL1A CDS | SEQ ID NO: 83 | GCTTGGGCCTATCTGGGTGG |
| TCL1A CDS | SEQ ID NO: 84 | GGCTTGGGCCTATCTGGGTG |
| TCL1A CDS | SEQ ID NO: 85 | CTTCTCCTCAGATAAAGGAT |
| TCL1A CDS | SEQ ID NO: 86 | CCCAGGCCCACAGGCGGTCC |
| TCL1A CDS | SEQ ID NO: 87 | CAGGCTTGGGCCTATCTGGG |
| TCL1A CDS | SEQ ID NO: 88 | CCTATCTGGGTGGGGGTCAT |
| TCL1A CDS | SEQ ID NO: 89 | CTCTCTGCCTCTCAGATTGA |
| TCL1A CDS | SEQ ID NO: 90 | CCCGGACCGCCTGTGGGCCT |
| TCL1A CDS | SEQ ID NO: 91 | GATCCTCAGACTCCAGTTTC |
| TCL1A CDS | SEQ ID NO: 92 | CACATGATAGGCAGCAGGCT |
| TCL1A CDS | SEQ ID NO: 93 | ACCCGGACCGCCTGTGGGCC |
| TCL1A CDS | SEQ ID NO: 94 | GCAGCAGGCTTGGGCCTATC |
| TCL1A CDS | SEQ ID NO: 95 | CTCCCCGAGTGTCGGGCACT |
| TCL1A CDS | SEQ ID NO: 96 | CAAATACACGAACTTCTCCC |
| TCL1A CDS | SEQ ID NO: 97 | CAAAGCTGGCTGTACCTCGA |
| TCL1A CDS | SEQ ID NO: 98 | CTTCTCCTCAGATAAAGGAT |
| TCL1A CDS | SEQ ID NO: 99 | GTTACGGGTGCTCTTGCGTC |
| TCL1A CDS | SEQ ID NO: 100 | GGTGGCGGCCGCATGCTGCC |
| TCL1A promoter | SEQ ID NO: 101 | CTTTATATCCGGGCAGCATG |
| TCL1A promoter | SEQ ID NO: 102 | CCCGCCCCGCCGCTTGGTGG |
| TCL1A promoter | SEQ ID NO: 103 | AGCATGCGGCCGCCACCAAG |
| TCL1A promoter | SEQ ID NO: 104 | CCTCCCGCCCCGCCGCTTGG |
| TCL1A promoter | SEQ ID NO: 105 | ATGCGGCCGCCACCAAGCGG |
| TCL1A promoter | SEQ ID NO: 106 | CGTCCTCCCGCCCCGCCGCT |
| TCL1A promoter | SEQ ID NO: 107 | TGCGGCCGCCACCAAGCGGC |
| TCL1A promoter | SEQ ID NO: 108 | GCGGCCGCCACCAAGCGGCG |
| TCL1A promoter | SEQ ID NO: 109 | GCCGCCACCAAGCGGCGGGG |
| TCL1A promoter | SEQ ID NO: 110 | CCGCCACCAAGCGGCGGGGC |
| TCL1A promoter | SEQ ID NO: 111 | CCACCAAGCGGCGGGGGGGG |
| TCL1A promoter | SEQ ID NO: 112 | CAAGCGGCGGGGGGGGAGGA |
| TCL1A promoter | SEQ ID NO: 113 | GCGGGGGGGGAGGACGGCCT |
| TCL1A promoter | SEQ ID NO: 114 | CGGGGGGGGAGGACGGCCTT |
| TCL1A promoter | SEQ ID NO: 115 | GGGGGGGGAGGACGGCCTTG |
| TCL1A promoter | SEQ ID NO: 116 | GCGGGAGGACGGCCTTGGGG |
| TCL1A promoter | SEQ ID NO: 117 | CGACCCGGGGTCCCGCCCCA |
| TCL1A promoter | SEQ ID NO: 118 | CGGGAGGACGGCCTTGGGGC |
| TCL1A promoter | SEQ ID NO: 119 | ACGGCCTTGGGGGGGGACCC |
| TCL1A promoter | SEQ ID NO: 120 | CGGCCTTGGGGGGGGACCCC |
| TCL1A promoter | SEQ ID NO: 121 | CTTGGGGGGGGACCCCGGGT |
| TCL1A promoter | SEQ ID NO: 122 | AAGGTCGTTCTCCCGACCCG |
| TCL1A promoter | SEQ ID NO: 123 | TTGGGGGGGGACCCCGGGTC |
| TCL1A promoter | SEQ ID NO: 124 | CAAGGTCGTTCTCCCGACCC |
| TCL1A promoter | SEQ ID NO: 125 | CCAAGGTCGTTCTCCCGACC |
| TCL1A promoter | SEQ ID NO: 126 | CCGGGTCGGGAGAACGACCT |
| TCL1A promoter | SEQ ID NO: 127 | CGGGTCGGGAGAACGACCTT |
| TCL1A promoter | SEQ ID NO: 128 | GGGTCGGGAGAACGACCTTG |
| TCL1A promoter | SEQ ID NO: 129 | TCGGGAGAACGACCTTGGGG |
| TCL1A promoter | SEQ ID NO: 130 | CTGCCTTACGCCCCGCCCCA |
| TCL1A promoter | SEQ ID NO: 131 | CGGGAGAACGACCTTGGGGC |
| TCL1A promoter | SEQ ID NO: 132 | GGGAGAACGACCTTGGGGCG |
| TCL1A promoter | SEQ ID NO: 133 | CGACCTTGGGGGGGGGCGTA |
| TCL1A promoter | SEQ ID NO: 134 | CTTGGGGCGGGGCGTAAGGC |
| TCL1A promoter | SEQ ID NO: 135 | GGGGGGGGCGTAAGGCAGGA |
| TCL1A promoter | SEQ ID NO: 136 | GGCGGGGCGTAAGGCAGGAT |
| TCL1A promoter | SEQ ID NO: 137 | GCGGGGCGTAAGGCAGGATG |
| TCL1A promoter | SEQ ID NO: 138 | GGGCGTAAGGCAGGATGGGG |
| TCL1A promoter | SEQ ID NO: 139 | GGCGTAAGGCAGGATGGGGC |
| TCL1A promoter | SEQ ID NO: 140 | GCGTAAGGCAGGATGGGGCG |
| TCL1A promoter | SEQ ID NO: 141 | GCAGGATGGGGGGGGGTTTG |
| TCL1A promoter | SEQ ID NO: 142 | CAGGATGGGGGGGGGTTTGT |
| TCL1A promoter | SEQ ID NO: 143 | AGGATGGGGGGGGGTTTGTG |
| TCL1A promoter | SEQ ID NO: 144 | GGATGGGGGGGGGTTTGTGG |
| TCL1A promoter | SEQ ID NO: 145 | GGCGGGGTTTGTGGGGGTCT |
| TCL1A promoter | SEQ ID NO: 146 | GGGGTTTGTGGGGGTCTTGG |
| TCL1A promoter | SEQ ID NO: 147 | GGGGGTCTTGGTGGCAAGTG |
| TCL1A promoter | SEQ ID NO: 148 | GGGGTCTTGGTGGCAAGTGA |
| TCL1A promoter | SEQ ID NO: 149 | GGCAAGTGAGGGTCCCGCGC |
| TCL1A promoter | SEQ ID NO: 150 | CCGGGACGTAGCGCCTGCGC |
| TCL1A promoter | SEQ ID NO: 151 | GCCGGGACGTAGCGCCTGCG |
| TCL1A promoter | SEQ ID NO: 152 | CCCGCGCAGGCGCTACGTCC |
| TCL1A promoter | SEQ ID NO: 153 | TCCAGGGAGCAAGTCAGGCC |
| TCL1A promoter | SEQ ID NO: 154 | TTCCAGGGAGCAAGTCAGGC |
| TCL1A promoter | SEQ ID NO: 155 | ACCGTTCCAGGGAGCAAGTC |
| TCL1A promoter | SEQ ID NO: 156 | TCCCGGCCTGACTTGCTCCC |
| TCL1A promoter | SEQ ID NO: 157 | GCCTGACTTGCTCCCTGGAA |
| TCL1A promoter | SEQ ID NO: 158 | CAGGAGCTGCCACCGTTCCA |
| TCL1A promoter | SEQ ID NO: 159 | CCAGGAGCTGCCACCGTTCC |
| TCL1A promoter | SEQ ID NO: 160 | TGACTTGCTCCCTGGAACGG |
| TCL1A promoter | SEQ ID NO: 161 | CCTGGAACGGTGGCAGCTCC |
| TCL1A promoter | SEQ ID NO: 162 | CTGGAACGGTGGCAGCTCCT |
| TCL1A promoter | SEQ ID NO: 163 | GGGGCCGGGTCTGCGTTCCC |
| TCL1A promoter | SEQ ID NO: 164 | AGCTCCTGGGAACGCAGACC |
| TCL1A promoter | SEQ ID NO: 165 | GGCGCGGGCCAGCTGGGGCC |
| TCL1A promoter | SEQ ID NO: 166 | AGGCGCGGGCCAGCTGGGGC |
| TCL1A promoter | SEQ ID NO: 167 | AACGCAGACCCGGCCCCAGC |
| TCL1A promoter | SEQ ID NO: 168 | AGGAAGGCGCGGGCCAGCTG |
| TCL1A promoter | SEQ ID NO: 169 | GAGGAAGGCGCGGGCCAGCT |
| TCL1A promoter | SEQ ID NO: 170 | CGAGGAAGGCGCGGGCCAGC |
| TCL1A promoter | SEQ ID NO: 171 | CGGGGCCTCGAGGAAGGCGC |
| TCL1A promoter | SEQ ID NO: 172 | CCGGGGCCTCGAGGAAGGCG |
| TCL1A promoter | SEQ ID NO: 173 | GCTGGCCCGCGCCTTCCTCG |
| TCL1A promoter | SEQ ID NO: 174 | GCTGGCCGGGGCCTCGAGGA |
| TCL1A promoter | SEQ ID NO: 175 | CGCTGCTGGCCGGGGCCTCG |
| TCL1A promoter | SEQ ID NO: 176 | CCGCGCCTTCCTCGAGGCCC |
| TCL1A promoter | SEQ ID NO: 177 | GTCGGTGTCGCTGCTGGCCG |
| TCL1A promoter | SEQ ID NO: 178 | GGTCGGTGTCGCTGCTGGCC |
| TCL1A promoter | SEQ ID NO: 179 | CGGTCGGTGTCGCTGCTGGC |
| TCL1A promoter | SEQ ID NO: 180 | GCGGCGGTCGGTGTCGCTGC |

shRNA, RNAi and anti-sense RNA agents: The anti-TCL1A agent may be an shRNA or an antisense oligonucleotide (ODN). Exemplary shRNA sequences are provided in Table 1. By RNAi agent is meant an agent that modulates expression by a RNA interference mechanism. The RNAi agents employed in one embodiment are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

Alternatively, an antisense sequence is complementary to the targeted RNA, and inhibits its expression. One or a combination of antisense molecules may be administered, where a combination may comprise multiple different sequences. Antisense molecules may be produced by expression of all or a part of the target RNA sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Anti-sense molecules of interest include antagomir RNAs, e.g. as described by Krutzfeldt et al. (2005) Nature 438:685-689, herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Genome editing. In some embodiments an anti-TCL1A agent utilizes a class 2 CRISPR/Cas effector protein (or a nucleic encoding the protein), e.g., as targeted endonuclease to alter the genomic sequence at the TCL1A locus in a manner that decreases expression of TCL1A. Exemplary guide RNAs may be found in Table 2. In class 2 CRISPR systems, the functions of the effector complex (e.g., the cleavage of target DNA) are carried out by a single protein (which can be referred to as a CRISPR/Cas effector protein) — where the natural protein is an endonuclease (e.g., see Zetsche et al, Cell. 2015 Oct. 22; 163(3):759-71; Makarova et al, Nat Rev Microbiol. 2015 Nov; 13(11):722-36; Shmakov et al., Mol Cell. 2015 Nov. 5; 60(3):385-97; and Shmakov et al., Nat Rev Microbiol. 2017 March; 15(3):169-182: “Diversity and evolution of class 2 CRISPR-Cas systems”). As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

A nucleic acid that binds to a class 2 CRISPR/Cas effector protein (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence, which is a nucleotide sequence that is complementary to a sequence of a target nucleic acid.

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Expression construct: Anti-sense, RNAi, etc. may administered as polynucleotides, e.g. oligonucleotides in a suitable delivery system, or may be introduced on an expression vector into a cell to be engineered. For example, a coding sequence may be introduced into a target cell using CRISPR technology. CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The nucleic acid encoding a polynucleotide agent is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors will contain a promoter that is recognized by the host organism and is operably linked to the desired sequence for expression. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Host cells, including hematopoietic stem cells, etc. can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

### Methods

The methods of the disclosure include administration of an agent, e.g. an anti-TCL1A agent, for the treatment or prevention of hematologic malignancies, which can provide for an additive and/or synergistic effect in the reduction of clonal and/or tumor cells. It is shown herein that increased expression of TCL1A is associated with increased clonal expansion. It has been found that down-regulating TCL1A can prevent clonal expansion.

In some embodiments, an individual identified as having CHIP is treated with an effective dose of an agent to reduce TCL1A expression or activity, i.e. an anti-TCL1A agent. In some such embodiments, hematopoietic stem cells of the individual are engineered to have reduced expression of TCL1A, e.g. by in vitro modification of the promoter of coding sequence of TCL1A to reduce expression; using CRISPR induced frameshifts to prevent the development of leukemia in those undergoing hematopoietic stem cell transplantation (HSCT), e.g. during genetic correction of autologous HSCs in sickle-cell disease; and the like. In some embodiments the individual is treated with an agent that reduces TCL1A expression, e.g. in circulating cells, in bone marrow, etc. Such an agent includes, without limitation, anti-sense oligonucleotides specific for TCL1A, RNAi agents specific for TCL1A, small molecule inhibitors of TCL1A activity, antibodies and antibody fragments specific for the inhibition of TCL1A, and the like. The treatment may be combined with administration of additional agents or regimens useful in the treatment of hematologic malignancies. The treatment can provide for prevention, i.e. a reduction in the development of hematologic cancers, including without limitation acute myeloid leukemia, myelodysplastic syndrome, myeloproliferative neoplasms, chronic myeloid leukemia, chronic myelomonocytic leukemia, and diffuse large B-cell lymphoma, as well as heart disease and death in persons with clonal hematopoiesis, who are at risk for these conditions.

In some embodiments, an individual selected for CHIP treatment is genotyped for SNP rs2887399 prior to treatment, and found to have the reference allele. In some embodiments an individual selected for CHIP treatment described herein is genotyped for the presence of a driver mutation in one or more of TET2, ASXL1, SF3B1, SRSF2, TP53, JAK2, PPM1 D, NRAS, KRAS, IDH1, and IDH2 prior to treatment, and found to have at least one such driver mutation.

The methods include administration of an agent, e.g. an anti-TCL1A agent, for the treatment or prevention of hematologic malignancies in combination therapies, which may provide for an additive and/or synergistic effect in the reduction of clonal and tumor cells. Specific combination therapies include, without limitation, combinations with cytoreductive agents and therapies, combinations with hypomethylating (epigenetic) agents, combinations with immuno-oncology agents, including those agents that act on T cells, combinations with tumor-targeted agents, for example antibodies that selectively bind to cancer cell markers, combinations with biologic factors that increase phagocytic cell activation, growth, localization and the like; combination with transplantation, transfusion, leukapheresis, erythropoietin stimulating agents including erythropoietin, and the like.

The methods include patient selection for efficacy of an agent for the treatment of hematologic malignancies and treatment of selected patients. Selection criteria may be based on clinical parameters, expression of biomarkers, and the like. Included as biomarkers are molecular mutations for enrichment of efficacy, e.g. CHIP associated driver genes, MDS-specific mutations, TCL1A genotyping, etc.

“In combination with”, “combination therapy” and “combination products” refer, in certain embodiments, to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

Chemotherapeutic agents that can be administered in combination with an anti-TCL1A agent include, without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination with an agent may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

An agent may be administered in combination with an immunomodulator, such as a cytokine, a lymphokine, a monokine, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TNF-α or TNF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and lymphotoxin (LT).

Tumor specific monoclonal antibodies that can be administered in combination with an agent may include, without limitation, gemtuzumab ozogamicin (Myelotarg), Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

Of particular interest are hypomethylating (also known as epigenetic) agents for combination with an agent. A hypomethylating agent is a drug that inhibits DNA methylation. Currently available hypomethylating agents block the activity of DNA methyltransferase (DNA methyltransferase inhibitors/DNMT inhibitors). Currently two members of the class, azacitidine and decitabine are FDA-approved for use in the United States. Guadecitabine is also of interest. Because of their relatively mild side effects, azacitidine and decitabine are particularly feasible for the treatment of older patients and patients with co-morbidities. Both drugs have remarkable activity against AML blasts with unfavorable cytogenetic characteristics.

Treatment of hematologic malignancies can be combined with one or more therapeutic entities. In some embodiments the additional therapeutic entity in an immune response modulator. Immune checkpoint proteins are immune inhibitory molecules that act to decrease immune responsiveness toward a target cell, particularly against a tumor cell in the methods of the invention. Endogenous responses to tumors by T cells can be dysregulated by tumor cells activating immune checkpoints (immune inhibitory proteins) and inhibiting co-stimulatory receptors (immune activating proteins). The class of therapeutic agents referred to in the art as “immune checkpoint inhibitors” reverses the inhibition of immune responses through administering antagonists of inhibitory signals. Other immunotherapies administer agonists of immune costimulatory molecules to increase responsiveness.

The immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279) —are both inhibitory receptors. The clinical activity of antibodies that block either of these receptors implies that antitumor immunity can be enhanced at multiple levels and that combinatorial strategies can be intelligently designed, guided by mechanistic considerations and preclinical models.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity. CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation.

Other immune-checkpoint proteins are PD1 and PDL1. Antibodies in current clinical use against these targets include nivolumab and pembrolizumab. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on TReg cells, where it may enhance their proliferation in the presence of ligand. Because many tumors are highly infiltrated with TReg cells, blockade of the PD1 pathway may also enhance antitumor immune responses by diminishing the number and/or suppressive activity of intratumoral TReg cells.

Lymphocyte activation gene 3 (LAGS; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy. Antibody targeting of these receptors can be used in the methods of the invention.

TIM3 inhibits T helper 1 (TH1) cell responses, and TIM3 antibodies enhance antitumor immunity. TIM3 has also been reported to be co-expressed with PD1 on tumor-specific CD8+ T cells. Tim3 blocking agents can overcome this inhibitory signaling and maintain or restore anti-tumor T cell function.

BTLA is an inhibitory receptor on T cells that interacts with TNFRSF14. BTLAhi T cells are inhibited in the presence of its ligand. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions. Dual blockade of BTLA and PD1 enhances antitumor immunity.

Agents that agonize an immune costimulatory molecule are also useful in the methods of the invention. Such agents include agonists or CD40 and OX40. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L). Stimulation through CD40 activates macrophages. Agonistic CD40 agents may be administered substantially simultaneously with agents; or may be administered prior to and concurrently with treatment with to pre-activate macrophages.

Agents that alter the immune tumor microenvironment are useful in the methods of the invention. Such agents include IDO inhibitors which inhibit the production of indoleamine-2,3-dioxygenase (IDO), an enzyme that exhibits an immunosuppressive effect.

Other immuno-oncology agents that can be administered in combination according to the methods described herein include antibodies specific for chemokine receptors, including without limitation anti-CCR4 and anti-CCR2. Anti CCR4 (CD194) antibodies of interest include humanized monoclonal antibodies directed against C-C chemokine receptor 4 (CCR4) with potential anti-inflammatory and antineoplastic activities. Exemplary is mogamulizumab, which selectively binds to and blocks the activity of CCR4, which may inhibit CCR4-mediated signal transduction pathways and, so, chemokine-mediated cellular migration and proliferation of T cells, and chemokine-mediated angiogenesis. In addition, this agent may induce antibody-dependent cell-mediated cytotoxicity (ADCC) against CCR4-positive T cells. CCR4, a G-coupled-protein receptor for C-C chemokines such MIP-1, RANTES, TARC and MCP-1, is expressed on the surfaces of some types of T cells, endothelial cells, and some types of neurons. CCR4, also known as CD194, may be overexpressed on adult T-cell lymphoma (ATL) and peripheral T-cell lymphoma (PTCL) cells.

The combination therapy described above may be combined with other agents that act on regulatory T cells, e.g. anti-CTLA4 Ab, or other T cell checkpoint inhibitors, e.g. anti-PD1, anti-PDL1 antibodies, and the like.

In some embodiments, administration of a combination of agents of the invention is combined with an effective dose of an agent that increases patient hematocrit, for example erythropoietin stimulating agents (ESA). Such agents are known and used in the art, including, for example, Aranesp® (darbepoetin alfa), Epogen®/Procrit® (epoetin alfa), Omontys® (peginesatide), Procrit®, etc. See, for example, U.S. Pat. No. 9,623,079.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer. Diagnostic Methods

In some embodiments, methods are provided for determining the clonal growth rate of a hematopoietic clone from a sample, e.g. a peripheral blood sample, using PACER (passenger-approximated clonal expansion rate). In some embodiments the determination is performed on a single sample, i.e. in the absence of a time course of samples. In some embodiments an individual is treating in accordance with the findings of the clonal growth determination, where treatment may comprise administration of an agent or regimen that reduces the number of cells in a clone.

The methods of determining clonal growth are based on sequence analysis of mutations present in the clone. While a clone, e.g. a clone of hematopoietic stem cells, accumulates mutations, most are passenger mutations that do not have any significant consequence on the stem cells ability to divide or proliferate. These passenger mutations are largely undetectable until the stem cell acquires a somatic mutation in a driver gene that provides the clone with a clonal advantage, e.g. mutations in one or more of DNMT3A, TET2, ASXL1, JAK2, etc.

DNA sequencing a peripheral blood sample from an individual with CHIP identifies CHIP driver mutations, and also identifies a body of passenger mutations. The number of passenger mutations is used to estimate clone age. As clonal hematopoiesis blood clones expand, the variant allele fraction of both driver and passenger mutations increases. It is shown that passenger mutations are likely to precede the driver mutation. As the passenger mutations accrue at a constant rate across time that is similar across individuals, they can be used to date the acquisition of the driver. For two individuals of the same age and with clones of the same size, the clone with more passenger mutations has greater growth potential, as it expanded to the same size in less time. Higher growth potential clones will harbor more detectable passengers than lower fitness clones that arose at the same time.

The number of passenger mutations in the founding cell of a CHIP clone is used to determine the date of acquisition of the driver mutation, which can be determined with whole genome sequencing of a sample from a single time-point. The number of passengers in any given cell is the sum of the mutations present prior to the acquisition of the driver event (ancestral) and mutations acquired after the driver event (sub-clonal). Detectable passengers in whole blood DNA are more likely to be ancestral passengers than sub-clonal passengers. Also, high fitness clones harbor more detectable passengers than lower fitness clones of the same age. Therefore, for two individuals of the same age and with clones of the same size, the clone with more passengers is expected to be more fit.

To estimate the number of passenger mutations, a cell population is sequenced to generate a database of sequence variants present in the sample. The initial database of sequence variants comprises a combination of true somatic variants, germline variants, and sequencing artifacts, and thus is filtered to provide a more accurate representation of passenger variants in the database. To filter, variants are selected that are found in a single individual in the dataset. Variants can be excluded that have a VAF of greater than 35%. Variants can be excluded that comprise only C>T and T>C mutations.

Driver mutations can be determined based on changes in the database of known CHIP driver genes. Clonal expansion is quantified clonal expansion by dividing the change in VAF by the change in time (years) (dVAF/dT) of driver variants identified in a sample. A simple estimator of dVAF/dT is designed using only the passengers, VAF, and age from the first blood draw. A model that included age and VAF in addition to passenger count improved the prediction of clonal expansion. These results show that inferring clonal expansion from age- and VAF-adjusted passenger mutation counts described past growth, but predicted future growth rate.

In some embodiments, the presence of passenger mutations in a hematopoietic sample from an individual suspected of having CHIP provides a composite measure of clone fitness and clone birth date, using the PACER method.

Genetic sequencing of the hematopoietic sample first identifies non-reference variants in the genomes using standard algorithms, selecting for variants that are present at variant allele frequencies below the threshold for a germline variant. To reduce the likelihood of recurrent sequencing artifacts, somatic variants that were found only in a single individual in the dataset are used. As different mutation sub-types varied in their association with age at blood draw, only C-T and T-C mutations are selected, as these were the most strongly age-associated. These steps provide identification of a set of variants in the genomes referred to as passengers. In some embodiments the steps are embodied as a program of instructions executable by computer and performed by means of software components loaded into the computer. The passenger count is then used to determine clone fitness and birth date. In some embodiments, the passenger count is compared to a reference sample, e.g. an individual with a known CHIP clone date and/or size.

Genotyping and/or detection, identification and/or quantitation of the genomic mutations can utilize sequencing. Sequencing can be accomplished using high-throughput systems. Sequencing can be performed using nucleic acids described herein such as genomic DNA, cDNA derived from RNA transcripts or RNA as a template. Sequencing may comprise massively parallel sequencing. In some embodiments, high-throughput sequencing involves the use of technology available by Helicos BioSciences Corporation (Cambridge, Massachusetts) such as the Single Molecule Sequencing by Synthesis (SMSS) method. In some embodiments, high-throughput sequencing involves the use of technology available by 454 Lifesciences, Inc. (Branford, Connecticut) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

In some embodiments, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 200401061 30; 20030064398; 20030022207; and Constans, A, The Scientist 2003, 17(13):36.

In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. Other high-throughput sequencing systems include those disclosed in Venter, J., et al. Science 16 February 2001; Adams, M. et al, Science 24 Mar. 2000; and M. J, Levene, et al. Science 299:682-686, January 2003; as well as US Publication Application No. 20030044781 and 2006/0078937. The growing of the nucleic acid strand and identifying the added nucleotide analog may be repeated so that the nucleic acid strand is further extended and the sequence of the target nucleic acid is determined.

The methods disclosed herein may comprise amplification of DNA. Amplification may comprise PCR-based amplification. Alternatively, amplification may comprise nonPCR-based amplification. Amplification of cfDNA and/or ctDNA may comprise using bead amplification followed by fiber optics detection as described in Marguiles et al. “Genome sequencing in microfabricated high-density pricolitre reactors”, Nature, doi: 10.1038/nature03959; and well as in US Publication Application Nos. 200200 12930; 20030058629; 20030 1001 02; 20030 148344 ; 20040248 161 ; 200500795 10,20050 124022; and 20060078909.

Amplification of the nucleic acid may comprise use of one or more polymerases. The polymerase may be a DNA polymerase. The polymerase may be a RNA polymerase. The polymerase may be a high fidelity polymerase. The polymerase may be KAPA HiFi DNA polymerase. The polymerase may be Phusion DNA polymerase. Amplification may comprise 20 or fewer amplification cycles. Amplification may comprise 20, 19, 18, 17, 16, 15, 14, 13, 12, 11, 10, or 9 or fewer amplification cycles. Amplification may comprise 18 or fewer amplification cycles. Amplification may comprise 16 or fewer amplification cycles. Amplification may comprise 15 or fewer amplification cycles.

The methods described herein may be performed by a computer program product that comprises a computer executable logic that is recorded on a computer readable medium. For example, the computer program can execute some or all of the following functions: (i) controlling isolation of nucleic acids from a sample, (ii) pre-amplifying nucleic acids from the sample or (iii) selecting, amplifying, sequencing or arraying specific regions in the sample, (iv) identifying and quantifying somatic mutations in a sample, (v) comparing data on somatic mutations detected from the sample with a predetermined threshold, and (vii) declaring an assessment of clonal growth.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of evaluating the clonal growth in an individual by accessing data that reflects the sequence of the selected clonal genomes from the individual, and/or the quantitation of one or more nucleic acids from the clonal genomes.

In one embodiment, the computer executing the computer logic of the invention may also include a digital input device such as a scanner. The digital input device can provide information on a nucleic acid, e.g., polymorphism levels/quantity.

In some embodiments, the invention provides a computer readable medium comprising a set of instructions recorded thereon to cause a computer to perform the steps of (i) receiving data from one or more nucleic acids detected in a sample; and (ii) diagnosing or predicting clonal growth based on the quantitation.

Kits may be provided. Kits may further include cells or reagents suitable for sequencing cells; and determining the passenger rates. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### TCL1A Alters Clonal Expansion in Blood

The size of a clone with a driver mutation has been implicated in modulating the severity of associated disease. In contrast to small clones, which are ubiquitous in older individuals and are benign, large clones are less common and more likely to result in hematologic malignancy and cardiovascular disease. Clonal expansion is the process by which a lineage of blood cells expands. Despite the malignancy of large clones, the molecular determinants of clonal expansion have been incompletely characterized. Using 5,551 CHIP carriers derived from 127,946 deep (38×) whole genomes from the NHLBI Transomics for Precision Medicine (TOPMed) initiative, we developed a sequencing based method for the prediction of clonal expansion from a single timepoint, validated using ultra-high depth (>300×) longitudinal sequencing. We then performed the first large-scale investigation of the germline determinants of clonal expansion. We anticipate that the identified molecular pathways will inform primordial diagnostic and therapeutic efforts for CHIP.

We identified high-confidence somatic mutations in peripheral blood by analyzing the TOPMed WGS with GATK Mutect2. To remove sequencing artifacts and germline variants we performed stringent variant filtering and quality control. We identified CHIP carriers using a curated list of leukemogenic driver mutations from driver genes (Methods). We identified 6,158 CHIP mutations in 5,551 individuals. As shown in our previous report, the prevalence of CHIP was strongly associated with age at blood draw, and >75% of these mutations were in DNMT3A, TET2, or ASXL1.

The variant allele fraction (VAF), defined as the proportion of sequencing reads at a locus containing the mutant allele, is an approximate measure of clone size. As the clone expands, the VAF of both the driver and passenger mutations increases. The number of passengers in any given cell is simply the sum of the mutations present prior to the acquisition of the driver event (founding passengers) and mutations acquired after the driver event (subclonal passengers). At VAF values of greater than 5-10%, the detectable passengers are far more likely to be founding passengers than subclonal passengers. This is because the subclonal passengers are private to each subsequent division of the original mutant cell, and, in the absence of second driver event, quickly fall below the limit of detection in bulk tissue. As the passengers accrue at a rate that is constant rate over time and that is similar between individuals, they can be used to date the acquisition of the driver. For two individuals of the same age and with clones of the same size, we expect the clone with more passengers to be more fit, as it expanded to the same size in less time, assuming the mutation rate in the two persons is the same. Furthermore, as the size of the clone also determines the number of detectable passengers from WGS, high fitness clones will harbor more detectable passengers than lower fitness clones that arose at the same time. Based on these observations, we used the detectable passengers as a composite measure of clone fitness and birth date.

To estimate the number of passengers, we first obtained Mutect2 variant calls from the whole genome for each CHIP carrier and a subset of people without detectable CHIP. As the raw variant calls are expected to contain a combination of true somatic variants, germline variants, and sequencing artifacts, we implemented a series of filters to enrich for the detection of true passengers. We first selected only those variants that were found in a single individual (singletons) in the dataset, as recurrent variants are enriched for germline polymorphisms and recurrent artifacts. We also excluded variants with a VAF greater than 35%, as these would be enriched for germline polymorphisms. As different base substitutions varied in their association with age at blood draw, we selected only C-T and T-C mutations, as these were the most strongly age-associated. On average, the CHIP carriers had 237 passengers (95% CI: 229-246), the median value was 206, and the maximum value was 16,279. Of the CHIP carriers, 90% had a single driver mutation. The passengers were enriched by 54% (95% Cl: 51%-57%) in the CHIP carriers compared to the controls after adjusting for age and study using a negative binomial regression. In the controls without CHIP, we presumed the passengers were incompletely removed artifacts or, in some people, reflective of unidentified clonal hematopoiesis. The passengers were also positively associated with age, on average increasing by 13.7% (95% Cl: 13.0%-14.3%) each decade.

We validated the passengers as an estimator of fitness theoretically and empirically. For the theoretical validation, we constructed a simulation of HSC dynamics to characterize the relationship between fitness and detectable passenger counts. We derived a hierarchical Bayesian latent-variable estimator of clone fitness (Methods) and confirmed its strong correspondence to the observed passenger counts. We estimated a passenger mutation rate per diploid genome per year of 2.3, or a per-base pair rate of 3.83e-10. Assuming 100,000 HSCs, this results in a per-base-pair passenger mutation rate of 3.83e-15 per HSC clone per year without correction for the sensitivity of the sequencing technology used.

To empirically validate the passengers, we used ultra-high depth sequencing in 80 CHIP carriers from the Women's Health Initiative (WHI) from two time points using single-molecule molecular inverse probe sequencing (smMIPS) targeted to the CHIP driver genes (Methods). We called somatic variants in these samples using an ensemble of VarScan, GATK Mutect2, and manual inspection through IGV (Methods). We defined clonal expansion by dividing the change in VAF by the change in time (years)

$$\frac{dVAF}{dT}$$

of the driver variants identified at the first blood draw. We constructed a simple estimator of

$$\frac{dVAF}{dT}$$

using only the passengers, VAF, and age from the first blood draw (Methods). This estimator predicted the inverse normal transformed

$$\frac{dVAF}{dT}$$

(Rsq=32.5%, Adjusted Rsq=28.6%, pvalue=1.5e-4). After adjusting for the passenger counts, age was negatively associated with

$$\frac{dVAF}{dT} ,$$

suggesting tnat clones acquired later in life were on average less fit than those acquired by younger individuals. We also observed that VAF at the first time point was negatively associated with

$$\frac{dVAF}{dT}$$

after adjustment for the other covariates, which may reflect the largest clones saturating in clonality.

Building on recent computational estimates of variant fitness, we estimated the distribution of passengers across the most common CHIP driver genes. We stratified the DNMT3A carriers by whether the driver mutation was a missense mutation at position 882 into DNMT3A R882+ and DNMT3A R882− carriers. We used DNMT3A R882− as a reference point and estimated the relative abundances of passengers in other genes using negative binomial regression adjusting for age and study. Consistent with previous reports, splicing genes (SF3B1, SRSF2, U2AF1) were the most enriched for passengers, followed by JAK2, and DNMT3A R882− was among the most depleted. Relative to the R882− carriers, we observed a modest enrichment of passengers in the R882+ carriers. These observations are concordant with prior empirical estimates of variant fitness derived from longitudinal sequencing of samples with clonal hematopoiesis.

To characterize the molecular pathways associated with clonal expansion, we performed a genome-wide association study (GWAS) of the inverse normal transformed passenger counts of CHIP carriers. We included age at blood draw, study, VAF, and the first ten genetic ancestry principal components, and used SAIGE to estimate the single variant association statistics among 19,913,304 variants. The GWAS identified a single locus at genome-wide significance at TCL1A. We used SuSIE to fine-map (Methods) a 200 kb region surrounding TCL1A which identified a credible set containing a single variant rs2887399. Each additional T allele was associated with a decrease in passenger count z-score by 0.15 (pvalue=4.5e-12). The alt-allele is common, occurring in 26% of TOPMed haplotypes. rs2887399 lies in a core promoter of TCL1A as defined by the Ensembl regulatory build 162 base-pairs from the canonical transcription start site (TSS) and in a CpG island. Analysis of the variant by the Open Targets variant-to-gene (V2G) function also nominated TCL1A as the causal gene. TCL1A has been implicated in prior reports as driver gene in lymphocytic malignancy.

We then asked whether any genetic variation associated with the passenger counts was specific to different CHIP mutations. We performed separate GWAS of passenger counts for carriers of TET2, DNMT3A, ASXL1, and splicing mutations. In TET2 carriers, we observed variation at the SASH1-UST locus was associated with passenger counts. The lead variant rs4897025 is a common (MAF=43%) intergenic variant that was associated with decreased passenger count burden (beta=−0.3, pvalue=2.8e-8). Previous reports have observed that downregulation of SASH1 is associated with increased risk for breast cancer. In DNMT3A carriers we observed no association between rs4897025 and passenger counts (pvalue=7.4e-1), consistent with its effect in the non-stratified passenger count GWAS (pvalue=1.4e-2). In TET2 carriers the effect size of alt-alleles of rs2887399 was larger than in the non-stratified GWAS (beta=−0.15 in non-stratified GWAS, beta=−0.24 in TET2 carrier GWAS). We observed no other germline variation that was associated with passenger counts in the other CHIP gene stratified GWAS, possibly due to limiting sample size.

We examined the association between the burden of rare variation with passenger counts in the 200 kb region surrounding TCL1A. We used the SCANG rare variant scan procedure to estimate the association, including all variants with a MAC<=300 (MAF<=3.7%). The SCANG procedure estimates the association between rare variants in moving windows across the genome and estimates the size of the windows. SCANG did not identify any regions at exome-wide significance (2.5e-06), though did identify one region within an order of magnitude (pvalue=6.6×10⁻⁶, family-wise pvalue=2×10⁻³). After conditioning on the rs2887399 genotypes in the rare variant analysis, the signal was attenuated, suggesting limited evidence for an independent rare-variant signal from rs2887399 in the same region (<1 Mb). We identified only 10 putative loss-of-function (pLOF) carriers of TCL1A TOPMed wide, so were underpowered to examine the burden of these variants.

We performed an expanded search of rare variation associated with the passengers. We used 1,698 genes associated with ‘cancer’ according to Open Targets to define variant groups. We performed SCANG association tests at every gene and its 150 Kb flanking region, including both coding and non-coding variants with a MAC<=300. We identified 15 windows associated with passenger counts at Bonferroni significance (pvalue=2.9×10 -5). We identified an intergenic region 113 kb from the TSS of TNFAIP3 (pvalue=5.4e-7) that is a distal enhancer of TNFAIP3 (GeneHancer). We also identified windows in between OLIG1 and OLIG2 (pvalue=7.9e-6) and 22 kb away from the HOX gene cluster (pvalue=4.6e-6).

As the allele frequency of rs2887399 varies by population, we asked whether passenger count was associated with the first two genetic ancestry principal components. We observed a positive association between values on the PC1 axis with singleton counts. Even after conditioning on the rs2887399 dosage, the association remained. A linear regression with rs2887399 dosage and the first two principal components as covariates explained 4% of the variation in the inverse-normal transformed passenger counts. Ancestry estimation using RFMix indicated a modest depletion of passengers in Sub-Saharan African genomes relative to European and East Asian genomes (Methods).

We asked whether the association between rs2887399 and passenger counts was modified by CHIP driver gene. Using DNMT3A as the reference, we investigated whether other genes had different effect estimates for rs2887399. We observed that alt-allele dosage in rs2887399 was more protective in TET2 than DNMT3A (beta=−0.23, pvalue=2×10⁻³), but we were underpowered to detect effects in other genes. These results suggest that the protective effects of rs2887399 vary by CHIP driver mutation and are weaker in DNMT3A than TET2. As the alt-homozygotes of rs2887399 were depleted for other CHIP mutations, we were underpowered to estimate the association between rs2887399 dosage and passenger counts in the other CHIP genes.

To further interrogate the interaction between rs2887399 and CHIP driver gene, we performed association tests between the variant and the acquisition of specific CHIP driver genes. In our previous analysis we reported that the T allele was associated with increased risk for DNMT3A mutation. In an expanded analysis of 74,974 individuals, we observed that rs2887399 is protective for non-DNMT3A mutations, including multiple non-DNMT3A driver mutations and splicing mutations. The alternate homozygous genotype was associated with decreased risk of acquisition of multiple (>1) non-DNMT3A mutations (OR=0.20, 95% CI: 0.06-0.51, Methods). These results indicate that rs2887399 increases risk for low fitness DNMT3A clones but is protective against clones that more strongly predict progression to frank hematologic malignancy, including JAK2, ASXL1, SRSF2, and SF3B1, and was especially protective against the acquisition of >1 non-DNMT3A driver mutations.

Previous analysis of blood cell indices in UK Biobank have implicated rs2887399 in reduced blood cell counts, consistent with altered hematopoiesis. To further characterize the disease associations of rs2887399, we performed a phenome-wide association study (PheWAS) lookup in UK Biobank. Although no genome-wide significant associations were identified among the case-control phenotypes, the alt-allele was nominally protective against myeloproliferative neoplasms (beta=−0.12, p value=2.7e-02) and leukemia (beta=−0.11, pvalue=1.0e-02). Previous reports have also identified that the alt-allele of rs2887399 increases risk for mosaic loss of the Y chromosome (beta=0.20, pvalue=6.0e-11), indicating a convergence of variation at the locus affecting multiple distinct clonal phenomena. A PheWAS lookup of gene-based test statistics using 45,596 UK Biobank exomes identified a nominal association between TCL1A coding variants with other anemias (UKB exome phewas, phecode 285, pvalue=2.3×10 2).

Next, we functionally characterized the rs2887399 locus. We first asked if the variant was associated with TCL1A expression in any cell type. As identified in the GTEx v8 eQTL release, the alt-allele reduces expression of TCL1A in whole blood (normalized effect size=−0.13, pvalue=1.4e-5). The association is likely driven by B-cells, as TCL1A is highly expressed in B-cells but appears to have absent or low expression in mature myeloid cells. We did not find evidence in the literature for expression of TCL1A in normal HSCs. We next asked whether CHIP associated mutations might alter the regulation of TCL1A in HSCs. Using a reference of chromatin accessibility in normal and pre-leukemic HSCs (pHSCs), we examined the ATAC-seq readout at the TCL1A promotor. Consistent with the lack of TCL1A transcripts in normal HSCs, we observed that the promoter was not accessible in either normal human donor HSCs, or pHSCs from patients with AML without any driver mutations. We also did not observe accessible chromatin in carriers of DNMT3A mutated pHSCs. In contrast, in the two patients with TET2 mutated pHSCs the TCL1A promoter was clearly accessible. These observations led us to propose the following mechanistic model: Normally, the TCL1A promoter is inaccessible and gene expression is low in HSCs. In the presence of driver mutations in TET2, ASXL1, SF3B1, SRSF2, JAK2, and possibly other genes, the TCL1A promoter opens, permitting gene expression and driving clonal expansion of the mutated cells. The presence of the alt-allele of rs2887399 prevents accessibility of chromatin at the TCL1A promoter, leading to reduced expression of TCL1A RNA, and abrogated clonal advantage due to the mutations.

Permitted by the analysis of the largest tranche of CHIP whole genomes to date, our results suggest several conclusions. The passenger counts represent a composite measure of the fitness and birth date of an underlying clone and provides a simple predictor of clonal expansion. Our results extend and apply recently developed theory on the evolutionary fitness of clones to permit estimation of fitness of a clone within a single individual. We used passenger counts in contrast to previous efforts which used the VAFs of driver variants. Despite being limited to a single blood draw, our estimates were concordant with those derived from empirical longitudinal investigations of clonal expansion in hematologic malignancy. Enabled by a method to estimate clonal expansion without serial sequencing, we mapped the associated molecular pathways.

We identified a common variant of large effect in the promoter of TCL1A associated with passenger counts, an oncogene that is deregulated in T-cell leukemia and lymphoma and that is a co-activator of Akt kinases. Activation of the Akt signaling pathway is associated with increased cell survival and proliferation. Our results suggest that regulation of TCL1A is also implicated in the acquisition of specific CHIP driver variants, where it was positively associated with risk of DNMT3A mutations, but negatively associated with other CHIP mutations. This suggests that TCL1A is not only associated with the fitness of CHIP clones, but also the type of CHIP mutation acquired.

Analysis of a chromatin accessibility atlas nominated a putative mechanism where the CHIP mutations differentially remodel chromatin at the TCL1A promoter. In contrast to a DNMT3A carrier and wild type, a TET2 mutation carrier had accessible chromatin at the TCL1A promoter. The accessible chromatin enables the effect of the rs2887399 alt-allele, which down-regulates TCL1A in HSCs. Future work is required to extend these chromatin accessibility atlases to carriers of other CHIP mutations.

Analysis of high-VAF passengers is key to the estimation of clonal expansion with this method, as it is only the passengers that occur on the predominant clone that is informative here. Both the clonal expansion method and the identified molecular pathways will inform diagnostic and therapeutic efforts for CHIP. Although our analysis has focused on passenger mutations in blood cells, our theory is not specific to the hematopoietic system, and may be informative in other tissues as well.

### Example 2

### Clonal Hematopoiesis is Driven by Aberrant Activation of TCL1A

A diverse set of driver genes, such as regulators of DNA methylation, RNA splicing, and chromatin remodeling, have been associated with pre-malignant clonal expansion of hematopoietic stem cells (HSCs). The factors mediating expansion of these mutant clones remain largely unknown, partially due to a paucity of large cohorts with longitudinal blood sampling. To circumvent this limitation, we developed and validated a method to infer clonal expansion rate from single timepoint data called PACER (passenger-approximated clonal expansion rate). Applying PACER to 5,071 persons with clonal hematopoiesis accurately recapitulated the known fitness effects due to different driver mutations. A genome-wide association study of PACER revealed that a common inherited polymorphism in the TCL1A promoter was associated with slower clonal expansion. Those carrying two copies of this protective allele had up to 80% reduced odds of having driver mutations in TET2, ASXL1, SF381, SRSF2, and JAK2, but not DNMT3A. TCL1A was not expressed in normal or DNMT3A-mutated HSCs, but the introduction of mutations in TET2 or ASXL1 by CRISPR editing led to aberrant expression of TCL1A and expansion of HSCs in vitro. These effects were abrogated in HSCs from donors carrying the protective TCL1A allele. Our results indicate that the fitness advantage of multiple common driver genes in clonal hematopoiesis is mediated through TCL1A activation. PACER is an approach that can be widely applied to uncover genetic and environmental determinants of pre-malignant clonal expansion in blood and other tissues.

To address the issue of collecting samples over time, we developed a method for approximating the rate of clonal expansion from a single timepoint, termed PACER, which was validated using longitudinal sequencing over 10 years in 55 CHIP carriers. We then used PACER to perform the first large-scale investigation of the germline determinants of clonal expansion in 5,071 CHIP carriers from the NHLBI Trans-Omics for Precision Medicine (TOPMed) program, which revealed activation of TCL1A as an event driving clonal expansion for multiple mutated genes in CHIP.

Derivation and validation of PACER. We identified high-confidence somatic mutations in peripheral blood DNA by analyzing TOPMed whole genome sequencing (WGS) data with Mutect2. To remove sequencing artifacts and germline variants we performed stringent variant filtering and quality control. We identified CHIP mutations in 5,071 individuals using a curated list of leukemogenic driver mutations (Methods). As described in our previous report, the prevalence of CHIP was strongly associated with age at blood draw, and >75% of these mutations were in DNMT3A, TET2, or ASXL1.

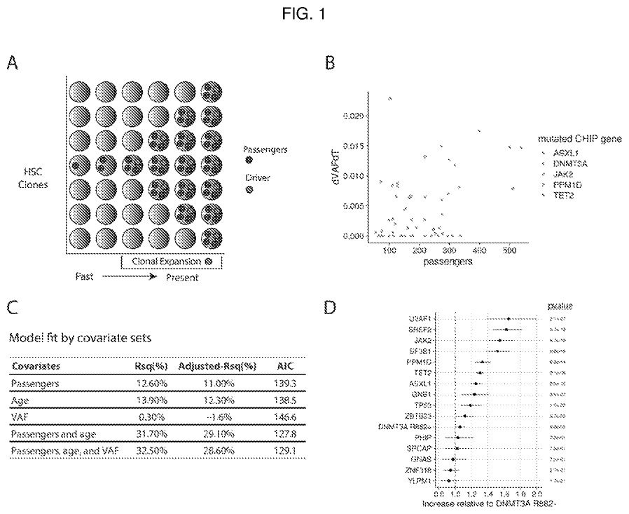

In HSCs, passenger mutations accrue at a rate that is fairly constant over time and that is similar across individuals. Thus, the number of passenger mutations in the founding cell of a CHIP clone can be used to approximate the date of acquisition of the driver mutation (FIG. 1a). Prior studies have enumerated passenger mutation burden in HSCs by performing WGS on colonies derived from single cells. We theorized that the passenger mutation burden in the founding cell for a CHIP clone could instead be approximated from WGS of whole blood DNA without isolation of single cells. As a mutant clone expands, the VAF of both the driver and passenger mutations increases. The number of passengers in any given cell is simply the sum of the mutations present prior to the acquisition of the driver event (ancestral passengers) and mutations acquired after the driver event (sub-clonal passengers). Because the limit of detection for mutations from WGS at ˜38× coverage depth is ˜8-10% VAF, the detectable passengers in whole blood DNA are far more likely to be ancestral passengers than sub-clonal passengers. This is because the sub-clonal passengers are private to each subsequent division of the original mutant cell, and, in the absence of a second driver event, quickly fall below the limit of detection in WGS data from bulk tissue. Furthermore, as the size of the clone also determines the number of detectable passengers from WGS due to the limited sensitivity of detection at 38× depth, high fitness clones will harbor more detectable passengers than lower fitness clones that arose at the same time. Based on these observations, we used the detectable passengers as a composite measure of clone fitness and birth date. For two individuals of the same age and with clones of the same size, we expect the clone with more passengers to be more fit, as it must have expanded to the same size in less time.

To estimate the number of passenger mutations, we first performed genome-wide somatic variant calling for 5,071 CHIP carriers and 23,320 controls without CHIP driver mutations. As these raw variant calls contain a combination of true somatic variants, germline variants, and sequencing artifacts, we implemented a series of stringent filters to enrich for the detection of true passengers (see Methods). We first selected only those variants that were found in a single individual in the dataset, as recurrent variants are enriched for germline polymorphisms and recurrent artifacts. We also excluded variants with a VAF greater than 35%, as these would also be enriched for germline polymorphisms. Since different base substitutions varied in their association with age at blood draw, we selected only C>T and T>C mutations, as these were the most strongly age-associated in our data, consistent with prior work identifying such mutations as essential elements of the “clock-like” signature.

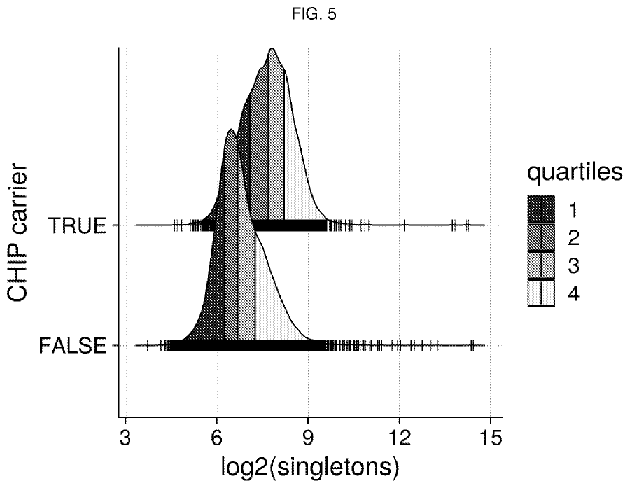

Amongst the 5,071 CHIP carriers, individuals had on average 271 passengers in WGS identified by our approach (interquartile range: 142-317). The passengers were increased by 54% (95% CI: 51%-57%) in the CHIP carriers (FIG. 5) compared to the controls after adjusting for age and study using a negative binomial regression. In the controls without CHIP, we presumed the detected passengers were reflective of clonal hematopoiesis without known driver mutations or due to drivers we could not assess such as mosaic chromosomal alterations (mCAs). Some of these could also have been incompletely removed artifacts. The passengers were also positively associated with age, on average increasing by 13.7% (95% CI: 13.0%-14.3%) each decade. Of the CHIP carriers in TOPMed, 89% had a single driver mutation.

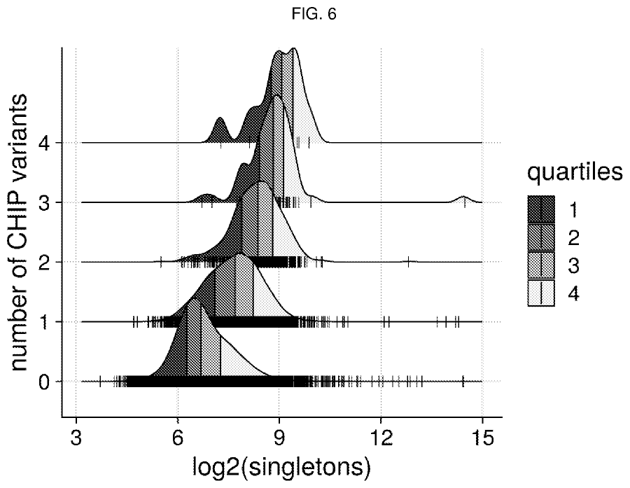

We found that each additional driver mutation detected in a given sample was associated with an increase in passenger mutation counts (FIG. 6). This is likely due to the presence of cooperating driver mutations in the same clone in these persons, as each successive expansion caused by a new driver mutation captures additional passenger mutations that accumulated in the time between the last driver event and the newer one. For this reason, we limited further analyses on clonal expansion rate only to the 4,536 CHIP carriers with a single driver event.

We validated the passengers as an estimator of fitness both theoretically and empirically. For the theoretical validation, we constructed a simulation of HSC dynamics to characterize the relationship between fitness and detectable passenger counts. The simulation indicated that founding passengers were associated with driver fitness (spearman ρ=0.09, pvalue<2×10⁻¹⁶). We estimated a passenger mutation rate per diploid genome per year of 2.3, or a per-base pair rate of 3.83×10⁻¹⁰. Assuming 100,000 HSCs, this results in a per-base-pair passenger mutation rate of 3.83×10⁻¹⁵ per HSC clone per year without correction for the sensitivity of the sequencing technology used. This number is substantially lower than previous estimates using WGS from single hematopoietic colonies, likely due the low sensitivity of detecting true passengers in whole blood DNA compared to the gold standard of single-cell derived colonies and also because we limited the base substitutions in our analysis to C>T or T>C. Nonetheless, we were able to use these data to derive a hierarchical Bayesian estimator of clone fitness (Methods), which adjusts for age at blood draw and cohort effects, and confirmed its correspondence to the observed passenger counts.

To empirically validate the predictive ability of passenger count, we performed targeted sequencing for driver variants from two blood samples taken approximately 10 years apart in 55 CHIP carriers from the Women's Health Initiative (WHI, Methods). WGS from the first time point was used to determine passenger count. We quantified clonal expansion by dividing the change in VAF by the change in time (years)

$$( \frac{dVAF}{dT} )$$

of the driver variants identified at the first blood draw. Of the sequenced carriers, 40 had clones with a single CHIP mutation that were constant in size or expanded. We constructed a simple estimator of

$$\frac{dVAF}{dT}$$

using only me passengers, VAF, and age from the first blood draw (Methods). Our theoretical framework considered passengers to be an estimate of clone fitness after accounting for age and VAF, hence these latter two variables were also considered in the model. A model only including VAF had lower predictive ability (Rsq=0.30%, Adjusted Rsq=−1.60%) for clonal expansion than a model only including passengers (Rsq=12.6%, Adjusted Rsq=11%). A model including only age had similar performance (Rsq32 13.9%, Adjusted Rsq=12.3%) to the passenger model. A model that included age and VAF in addition to passenger count improved the prediction of clonal expansion (Rsq=32.5%, Adjusted Rsq=28.6%, FIG. 1 b, c). These results suggested that inferring clonal expansion from age- and VAF-adjusted passenger mutation counts was able to not only describe past growth, but also predict future growth rate. We termed this approach PACER (passenger-approximated clonal expansion rate).

PACER predicts fitness of distinct driver mutations. Building on recent computational estimates of variant fitness, we estimated the distribution of passenger counts for the most common CHIP driver genes. We used non-R882 DNMT3A mutations as a reference point and estimated the relative abundances of passengers in other genes using negative binomial regression adjusting for age, VAF, and study. Mutations in splicing factors (SF3B1, SRSF2, U2AF1) and JAK2 V617F mutations were the fastest growing according to PACER, while DNMT3A R882− was among the slowest (FIG. 1d). Mutations in TET2, ASXL1, PPM1D, TP53, ZBTB33, and GNB1 were in the next tier and had approximately the same level of fitness estimated from PACER. Relative to the R882− carriers, we observed a modest increase in fitness in DNMT3A R882 mutant clones. These observations are concordant with prior empirical estimates of variant fitness derived from longitudinal sequencing of samples with clonal hematopoiesis and provides further validation of our approach.

Genome wide association study identifies inherited determinants of clonal expansion. We performed a genome-wide association study (GWAS) of PACER in CHIP carriers to identify inherited genetic variation that associates with clonal expansion. Association analyses were performed using the SAIGE statistical package. We included age at blood draw, study, VAF, and the first ten genetic ancestry principal components as covariates.

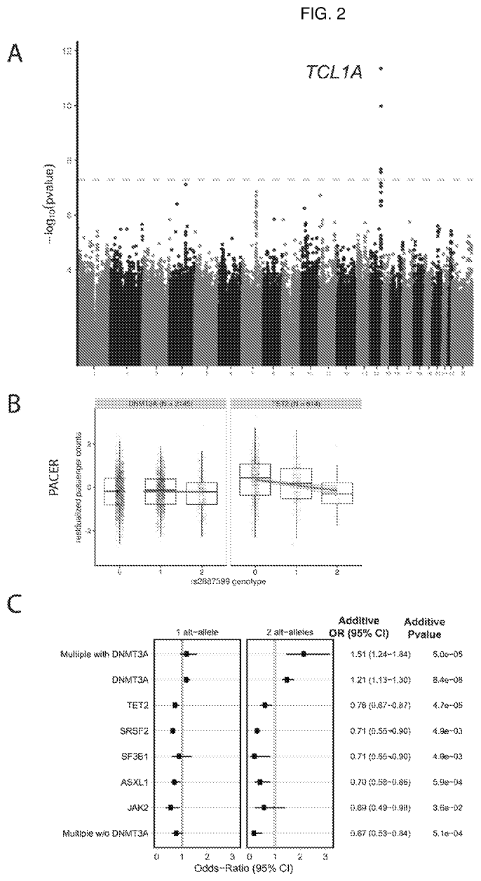

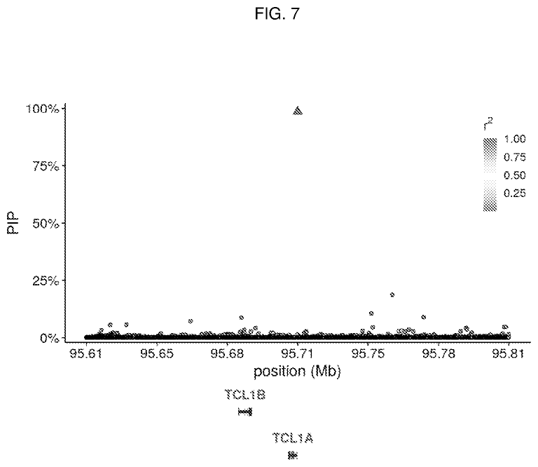

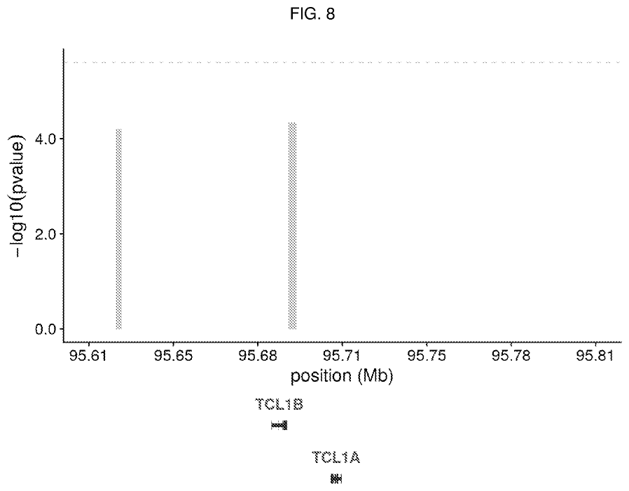

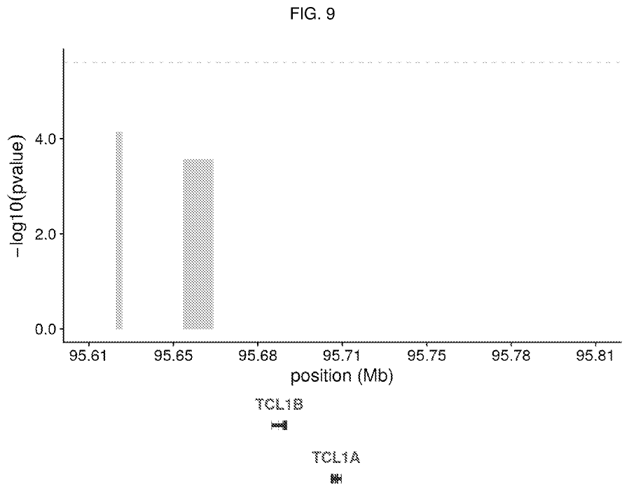

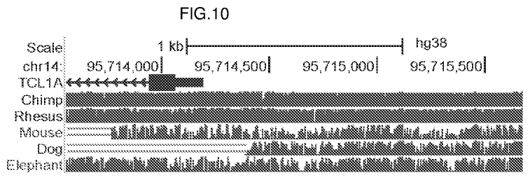

The GWAS identified a single locus at genome-wide significance overlapping TCL1A (FIG. 2a). We used SuSIE to perform genetic fine-mapping to identify the most likely causal set of variants, which further narrowed down the associated region to a credible set containing a single variant, rs2887399 (FIG. 7). Each additional alternative (alt) allele (T) was associated with a 0.15 decrease in passenger count z-score (pvalue=4.5×10⁻¹²). The alt-allele is common, occurring in 26% of haplotypes sequenced in TOPMed. rs2887399 lies in the core promoter of TCL1A as defined by the Ensembl regulatory build, 162 base-pairs from the canonical transcription start site (TSS) and in a CpG island. Analysis of the variant by the Open Targets variant-to-gene prediction algorithm also nominated TCL1A as the causal gene. We did not find any association between PACER and rare variants near rs2887399, suggesting that rs2887399 is not tagging other genetic variants and is the causal variant at this locus (FIG. 8-9). TCL1A has been implicated in lymphoid malignancies as a translocation partner in T-prolymphocytic leukemia, but it has not been studied in the context of HSC biology. TCL1A is also the only gene in the duplicated region of chromosome 14q32 associated with an inherited predisposition to develop myeloid malignancies shared by all kindreds. Of note, the region in the TCL1A promoter where rs2887399 resides is only partially conserved between humans and other primates, and poorly conserved with non-primate species (FIG. 10).

We next performed a genome-wide search of rare variation associated with the passengers. We identified 15 windows associated with passenger counts at Bonferroni significance (pvalue=2.9×10⁻⁵). We identified an intergenic region 113 kb from the TSS of TNFAIP3 (pvalue=5.4×10⁻⁷) that is a distal enhancer of TNFAIP3 (GeneHancer).

Association of rs2887399 with specific driver genes. We asked whether the association between rs2887399 and PACER was modified by CHIP driver gene. Using DNMT3A as the reference, we investigated whether other genes had different effect estimates for rs2887399. We observed that alt-allele dosage in rs2887399 was more protective against clonal expansion in TET2 than DNMT3A (beta=−0.24, pvalue=9.6×10⁻⁴, FIG. 2b).

Clones with a decreased expansion rate may never grow large enough to be detected, so we also performed association tests between rs2887399 and presence of a CHIP-associated driver mutation stratified by gene. In our previous analysis, we reported that the alt-allele was associated with increased risk for DNMT3A mutations. Prior reports have also identified that the alt-allele of rs2887399 decreases risk for mosaic loss of the Y chromosome (LOY) (OR=0.80, pvalue=4.3×10⁻¹³⁶). Here, we observed that rs2887399 was associated with significantly reduced odds of mutations in TET2, ASXL1, SF3B1, SRSF2, and possibly JAK2 (FIG. 2c). The effect size of rs2887399 was large for a common variant, as those carrying 2 copies of the alt-allele had odds ratios for having a driver mutation in these genes ranging from 0.22 to 0.63 (FIG. 2d). The risk reduction was particularly strong for mutations in SF3B1 and SRSF2, as well as for having >1 non-DNMT3A driver mutations (FIG. 2c-d, Methods). The latter group is particularly relevant clinically, as these persons have a high risk of risk of transformation, and in some cases may already have early-stage MDS. In sum, these results indicate that the alt-allele at rs2887399 is protective against CHIP due to driver mutations in several genes that have higher risk of progression to frank hematologic malignancy.

Previous analyses in UK Biobank have also implicated rs2887399 in reduced blood cell counts, consistent with an effect on hematopoiesis, but it is unknown if this is independent of hematological malignancy or CHIP.

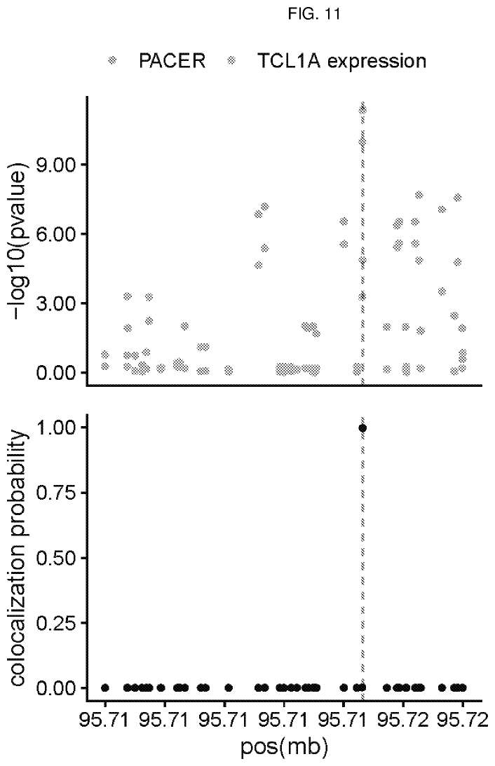

TCL1A expression in hematopoietic cells. Next, we sought to establish how rs2887399 might shape the hematologic phenotypes observed. We first asked if the variant was associated with TCL1A expression in any cell type. As identified in the GTEx v8 eQTL release, the alt-allele reduces expression of TCL1A in whole blood (normalized effect size=−0.13, pvalue=1.4×10⁻⁵ s). The GWAS of PACER colocalized with cis-expression quantitative trait loci (eQTLs) for TCL1A in whole blood (posterior probability of a single shared causal variant=97.1%, FIG. 11). The association in whole blood is likely driven by B-cells, as TCL1A is highly expressed in B-cells but appears to have absent or low expression in all other cell types in blood except for rare plasmacytoid dendritic cells (FIG. 5).

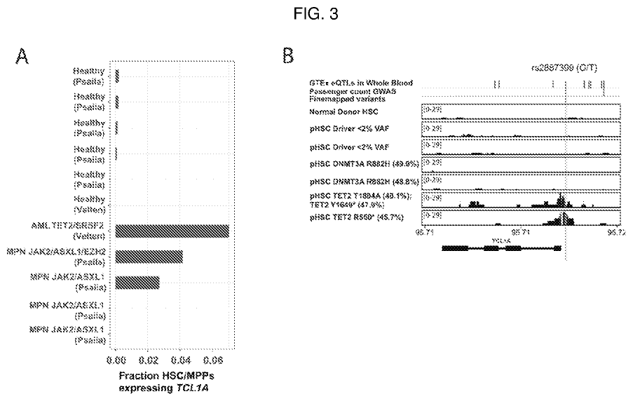

Little is known about TCL1A expression in HSCs. We examined whether CHIP-associated mutations altered the regulation of the TCL1A locus in human hematopoietic stem and progenitor cells (HSPCs) using publicly available single-cell RNA sequencing (scRNAseq) and ATAC-sequencing (ATAC-seq) datasets of normal and malignant hematopoiesis. TCL1A was expressed in fewer than 1 in 1000 cells identified as HSC/MPPs in scRNAseq data from 6 normal human marrow samples (range 0-0.17%). In contrast, TCL1A was expressed in a much higher fraction of HSC/MPPs in 3 out of 5 samples from persons with TET2 or ASXL1-mutated myeloid malignancies (range 2.7-7%) (FIG. 3a). Next, using a dataset of ATAC-seq in normal and pre-leukemic HSCs (pHSCs), we evaluated chromatin accessibility at the TCL1A promotor. Consistent with the lack of TCL1A transcripts in normal HSCs, we observed that the promoter was not accessible in either normal human donor HSCs or in HSCs from patients with AML that were not part of the mutant clone. We also did not observe accessible chromatin in two carriers of DNMT3A mutated pHSCs. In contrast, the two patients with TET2 mutated pHSCs had clearly accessible chromatin at the TCL1A promoter (FIG. 3b).

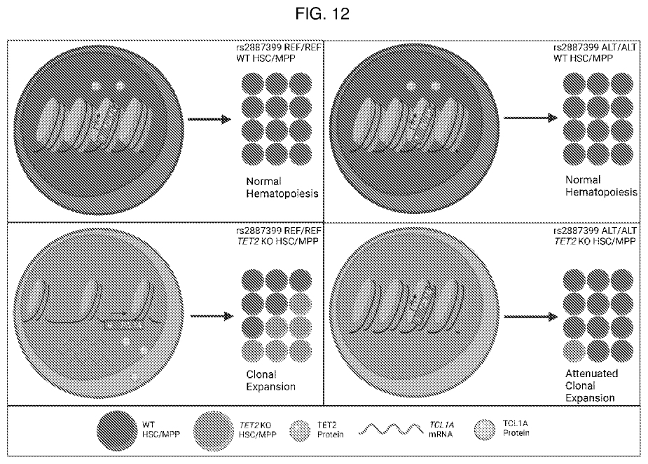

Functional effect of rs2887399 on normal and CHIP-mutated HSCs. These observations led us to propose the following mechanistic model: Normally, the TCL1A promoter is inaccessible and gene expression is absent or very low in HSCs. In the presence of driver mutations in TET2, ASXL1, SF381, SRSF2, or LOY, TCL1A is aberrantly expressed and drives clonal expansion of the mutated HSCs. The presence of the alt-allele of rs2887399 inhibits accessibility of chromatin at the TCL1A promoter, leading to reduced expression of TCL1A RNA and protein and abrogation of the clonal advantage due to the mutations (FIG. 12).

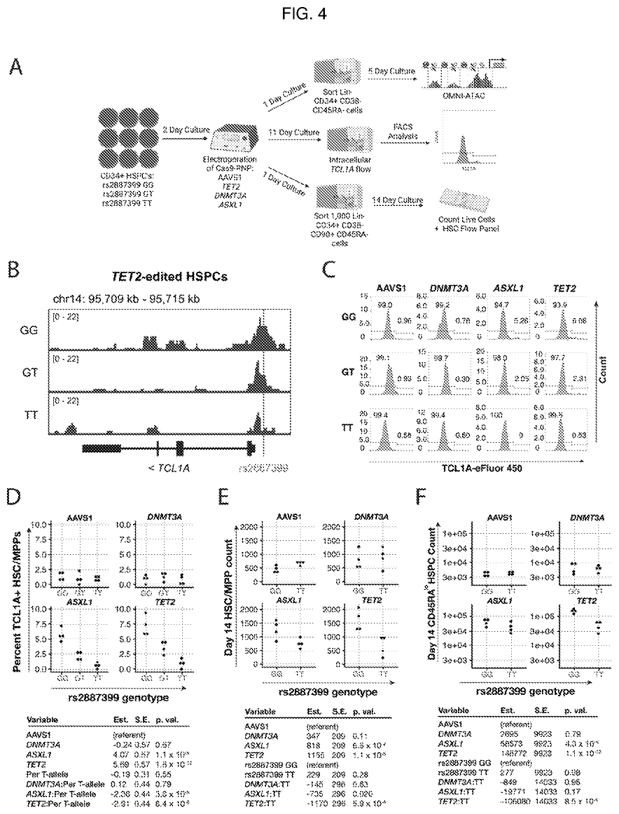

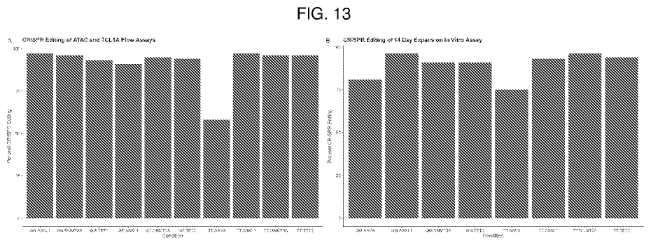

To test our model experimentally, we first obtained human CD34+ mobilized peripheral blood cells from donors who were GG (homozygous reference), TT (homozygous alternate), or GT (heterozygous) genotype at rs2887399. The three donors were healthy and between 29-32 years old at the time of donation. To mimic CHIP-associated mutations, we used CRISPR to introduce insertion-deletion mutations in DNMT3A, TET2, or ASXL1 in HSPCs for each rs2887399 genotype. Editing at the adeno-associated virus integration site 1 (AAVS1) was done as a control for each rs2887399 genotype (FIG. 4a). High efficiency of editing was confirmed by Sanger sequencing (FIG. 13).

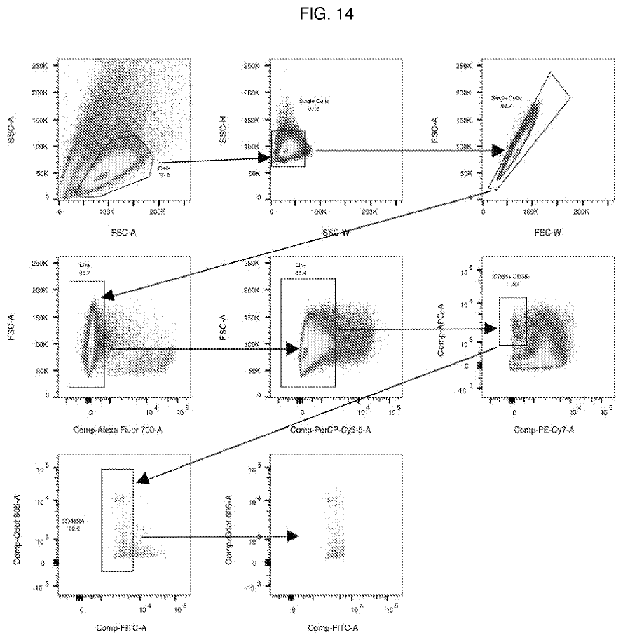

First, we examined whether the accessibility of the TCL1A promoter seen in the setting of TET2 mutations was altered by rs2887399 genotype. We edited bulk CD34 cells from each genotype for TET2, sorted cells with a marker profile of HSCs and multipotent progenitors (MPPs) (Lineage− CD34+ CD38−CD45RA−), cultured them for 5 days in cytokine-supported media, and then performed ATAC-seq (FIG. 14). Consistent with the pre-leukemic HSC data, we detected accessibility at the TCL1A promoter in TET2-edited cells from the rs2887399 GG donor. However, accessibility at the TCL1A promoter was decreased in the TET2-edited cells in samples from carriers of the T allele in a dose-dependent manner, indicating that the protective effect of the alt-allele of rs2887399 is mediated by blocking promoter accessibility (FIG. 4b). These results also suggest that alterations in the chromatin profile of HSPCs can occur within days after the introduction of a TET2 mutation.

Next, we asked if the differential chromatin accessibility due to rs2887399 altered TCL1A protein expression in HSCs/MPPs. We edited CD34+ cells from donors with the three rs2887399 genotypes at AAVS1, DNMT3A, TET2, and ASXL1. After 11 days in culture, we performed a flow cytometry-based assay for TCL1A protein expression. We found that -1% of HSCs/MPPs from AAVS1 or DNMT3A edited samples were positive for TCL1A, which did not vary by rs2887399 genotype. In contrast, 4.6-9.3% of HSCs/MPPs from the GG donor that had been edited for ASXL1 or TET2 expressed TCL1A, and the proportion of TCL1A positive HSC/MPPs decreased in donor samples with each additional T allele (4 biological replicates per condition) (FIG. 4c-d). There was minimal expression of TCL1A in any non-HSC/MPP CD34+ population in any of the samples. Notably, the proportion of TCL1A expressing HSC/MPPs was less than 10% in all samples even though the proportion of mutant cells was >90% (FIG. 14). This suggests that even in the presence of driver mutations in TET2 or ASXL1, only a fraction of HSC/MPPs are capable of expressing TCL1A at any given time and is consistent with the single-cell RNA sequencing data from hematological malignancy samples (FIG. 3b).

Finally, we asked if rs2887399 had any effect on expansion of HSPCs in vitro. For this experiment, we edited the CD34+ cells from GG and TT donors, sorted HSCs (Lin− CD34+ CD38− CD45RA− CD90+), and allowed the cultures to grow for 14 days, at which time cells were counted and analyzed for HSPC markers by flow cytometry. There was a notable expansion of cells bearing markers of HSCs/MPPs in the ASXL1 and TET2 edited samples from the rs2887399 GG donor compared to the AAVS1 edited sample, but this effect was abrogated in edited samples from the rs2887399 TT donor. A population of cells that was Lin−/lo CD34+ CD38− CD45RA dim (CD45RA^{dim} HSPCs), presumably progenitors descended from the HSC/MPP population, was also markedly expanded in the ASXL1 and TET2 edited samples from the GG donor, but the degree of expansion was partially reversed in the edited samples from the TT donor. There were no differences in any populations in the AAVS1 or DNMT3A edited samples based on rs2887399 genotype (4 biological replicates per condition) (FIG. 4e-f). Thus, carrying the alt-allele of rs2887399 abrogates the clonal expansion of HSPCs with ASXL1 and TET2 mutations in an experimental system.

Here, we have developed a novel method that allows us to infer clonal expansion rate from a single time point. Our results extend and apply recently developed theory on the evolutionary fitness of clones to permit estimation of fitness within a single individual. Unlike prior methods which used the VAFs of driver variants to estimate fitness, our development of a fitness estimator based on passenger mutations counts permits us to perform association tests for other factors associated with clonal expansion, such as inherited genetic variation and environmental exposures.

We performed the first ever GWAS for determinants of clonal expansion rate and identified a common variant of large effect in the promoter of TCL1A as the top hit. Remarkably, this single variant, which has previously been linked to reduced risk of LOY, was also associated with protection from driver mutations in TET2, ASXL1, SF381, SRSF2, and possibly JAK2. We also demonstrated with experimental work that TCL1A was normally lowly expressed in HSCs, but that the introduction of mutations in TET2 or ASXL1 led to expression of the protein, possibly by permitting promoter chromatin accessibility and hence transcription of the gene. This was completely prevented by the alt-allele of rs2887399, explaining the reduction in predicted clonal expansion rate by PACER and decreased prevalence of these driver mutations in those carrying the allele. To our knowledge, TCL1A itself is not somatically mutated in CHIP, perhaps because gain-of-function point mutations are not directly possible. How TCL1A expression causes clonal expansion of HSCs is an important question for future studies, but could be related to its reported role in AKT activation. Importantly, our results show that pharmacologically targeting TCL1A may suppress growth of CHIP and hematological cancers associated with mutations in these genes.

The large protective effect seen with rs2887399 suggests that TCL1A expression is likely a dominant factor mediating clonal expansion due to these mutations. This was especially the case for driver mutations in SRSF2 and SF3B1 which were very rare in those homozygous for the alt-allele, suggesting that activation of TCL1A expression may be a near requirement for clonal expansion due to these mutations. We do not explore how splicing factor mutations are mechanistically linked to TCL1A activation in this study, but one possibility is that mutations in SF3B1 or SRSF2 lead to a cryptic splice junction within the TCL1A 3′ UTR, which may lead to increased stability of the transcript in HSCs . These results may also potentially explain why mutations in Asx11, Sf3b1, and Srsf2 in mouse HSCs do not lead to robust clonal expansion, as the regulatory elements and non-coding regions of the mouse Tcl1 gene are not well conserved with human TCL1A. We previously reported that the alt-allele of rs2887399 was associated with increased risk of DNMT3A mutations, but here we found that carrying the alt-allele did not increase expansion rate of DNMT3A clones by PACER or result in increased expansion of DNMT3A edited HSPCs in vitro. One explanation for these discordant results is that the relative reduction in fitness advantage for other non-DNMT3A drivers in carriers of the alt-allele permits more opportunity for low fitness DNMT3A mutant clones to expand as hematopoiesis becomes more oligoclonal with aging. Alternatively, the interaction of DNMT3A mutations with TCL1A genotype may not be apparent in middle-aged or older persons, as it has recently been shown that the fitness of DNMT3A mutant clones declines with age.

In summary, we developed a novel tool for inferring clonal expansion rate and used it to identify TCL1A as a factor underlying the clonal fitness advantage of several driver mutations in CHIP. PACER is a powerful approach for identifying the genetic and environmental factors mediating clonal expansion in humans at population scale and may be applied to any tissue where pre-malignant clones exist.

### Methods

Study Samples. Whole genome sequencing (WGS) was performed on 127,946 samples as part of 51 studies contributing to Freeze 8 NHLBI TOPMed program as previously described. None of the TOPMed studies included selected individuals for sequencing because of hematologic malignancy. Each of the included studies provided informed consent. Age was obtained for 82,807 of the samples, and the median age was 55, the mean age 52.5, and the maximum age 98. The samples have diverse reported ethnicity (40% European, 32% African, 16% Hispanic/Latino, 10% Asian).

WGS Processing, Variant Calling and CHIP annotation. BAM files were remapped and harmonized through the functionally equivalent pipeline. SNPs and indels were discovered across TOPMed and were jointly genotyped across samples using the GotCloud pipeline. An SVM filter was trained to discriminate between high- and low-quality variants. Variants were annotated with snpEff 4.3. Sample quality was assessed through mendelian discordance, contamination estimates, sequencing converge, and among other quality control metrics.

Putative somatic SNPs were called with GATK Mutect2, which searches for sites where there is evidence for alt-reads that support evidence for variation, and then performs local haplotype assembly. We used a panel of normals to filter sequencing artifacts and used an external reference of germline variants to exclude germline calls. We deployed this pipeline on Google Cloud using Cromwell.

As described in our previous report, samples were annotated as having CHIP if the Mutect2 output contained at least one variant in a curated list of leukemogenic driver mutations with at least three alt-reads supporting the call. We expanded the list of driver mutations to include those in recently identified CHIP genes, increasing the number of CHIP cases from our previous report.

We called somatic singletons by identifying somatic variants that appeared in a single individual among the CHIP carriers and 23,320 additional controls for a total of 28,391 individuals. We excluded any variant that appeared in the TOPMed Freeze 5 germline call set (463 million variants). We excluded variants with a depth below 25 or above 100 and excluded any variants in low complexity regions or segmental duplications, as these are challenging for variant calling. We only included somatic singletons that were aligned to the primary chromosomal contigs. We excluded any variant with a VAF exceeding 35% as these may be enriched for germline variants that were not included in our other filters. We used cyvcf2 to parse the Mutect2 VCFs and encoded each variant in an int64 value using the variant key encoding. We developed a bespoke Python application to perform the singleton identification and filtering.

A special approach was required to identify somatic variants in U2AF1 since an erroneous segmental duplication in the region of the gene in the hg38 reference genome resulted in a mapping score of zero during alignment of the FASTQ file. We developed a Rust-HTSLIB binary to specifically identify reads associated with the U2AF1 variants S34F, S34Y, R156H, Q157P, and Q157R. A minimum of 5 alternate reads was required to include a variant in the somatic set of CHIP calls. The variant set was judged to have a high likelihood of being somatic based on the strong age association for persons carrying mutations as well as a high rate of co-mutation with other known drivers. The VAF was estimated by dividing the alternate read count by the total read count for U2AF1.

Amplicon sequencing validation. Targeted sequencing of the CHIP driver genes from 80 samples from the Women's Health Initiative (WHI) was performed using single-molecule molecular inversion probe sequencing (smMIPS). Reads were aligned with bwa-mem and processed with the mimips pileline. We called somatic variants using an ensemble of VarScan, Mutect2, and manual inspection with IGV.

Single Variant Association. Single variant association for each variant in the TOPMed Freeze 8 germline genetic variant call set with a MAC >20 was performed with SAIGE using the TOPMed Encore analysis server. To identify associations between rs2887399 and the acquisition of specific CHIP mutations, we used the same methods as our previous report on an analysis set of 74,974 individuals, including 4,697 cases and 70,277 controls. Age, genotype inferred sex, the first ten genetic ancestry principal components, and study were included as covariates.

We performed SAIGE single variant association analyses on the passengers including age at blood draw, sex, VAF, study, and the first ten genetic ancestry principal components as covariates. We applied an inverse normal transformation to the passenger counts. We declared variants from this analysis as significant if their p-value was less than 5×10⁻⁸.

Estimation of association between rs2887399 genotypes and CHIP mutation acquisition. We coded the rs2887399 genotypes as a categorical variable rather than a linear quantitative coding to estimate effects separately for the heterozygotes and the alt-homozygotes using the ref-homozygotes as the reference level. We estimated the associations using firth logistic regression to reduce bias in estimation resulting from low cell counts, and included age, genotype inferred sex, and the first ten genetic ancestry components as covariates.

Fine-mapping of the TCL1A region. We applied the SuSIE algorithm to the genotypes included in a 200 kb region surrounding TCL1A. We used the same covariates as the single variant association analysis. We used the posterior inclusion probabilities (PIP) and credible sets identified by SuSIE to identify the putative causal variant. We used LD directly calculated on the genotypes as opposed to an external reference.

Rare Variant Analyses. We performed gene-based tests on 1,698 cancer associated genes their flanking regions using the SCANG procedure. We identified these genes by downloading the targets associated with cancer in Open Targets, and then filtered to include only genes with an association score of 1.0. The most prevalent CHIP driver genes were included among this list. We used the inverse normal transformed passenger counts as the phenotype with the same covariates as before. We specified the minimum size of the grouped regions as 30 variants and the maximum as 200. We included all PASS variants with a minor allele count greater than four and less than 300 (MAF of 3.7% in the analyzed samples). We parsed the genotypes using cyvcf2 and stored them as dgCMatrix using the Matrix package from the R 3.6.1 programming language.

We set the p-value filter to calculate SKAT test-statistics at 5×10⁻⁴. We did not group the variants by annotation and we declared regions as significant if their pvalue was less than 2.9×10⁻⁵ (0.05/1,698). We controlled for relatedness by incorporating a sparse kinship matrix as estimated by the PC-AiR method from the GENESIS R package. We specified separate residual variance terms for each study to control for heterogeneous residual variance. We grouped together all studies where the number of analyzed samples was less than 200.

Enrichment of passengers by driver gene. We estimated the association between the driver genes and the passenger counts using DNMT3A as the reference in a negative binomial regression using the glm.nb function from the MASS R package. We included age, study, VAF, and sex as covariates. We included driver genes with at least 30 mutations and reported genes that had a different effect relative effect than DNMT3A if the pvalue of the coefficient was less than 1×10⁻².

Estimation of passenger mutation rate, clone fitness, and clone birth date. We developed a hierarchical Bayesian latent variable model using the Stan probabilistic programming language. We used the negative binomial likelihood with a mean and overdispersion parameterization to facilitate interpretation. We used the identity function to link the passenger counts to the predictors as we modeled the effects on an additive scale. We modeled the expectation and overdispersion of the passenger counts observed at time (tᵢ) as

E(countsᵢ(tᵢ))=μTᵢ+sᵢ(tᵢ−Tᵢ)+αₖ

countsᵢ(tᵢ)−NB(E(countsᵢ(tᵢ)), I(i∈CHIP)θ₀+(1−I(i∈CHIP))θ₁)

Where Tᵢ is the time of the driver acquisition for sample i with a blood draw at time tᵢ, μ is the mutation rate per diploid genome per year for the HSC population, sᵢ is the fitness of the clone, and αₖ represents a study specific random intercept for sample i included in study k. We can interpret tᵢTᵢ as the lifetime of the clone in years. We used a negative binomial likelihood as there was overdispersion relative to a Poisson distribution.

We included several constraints and priors on the parameters to make them identifiable. We constrained Tᵢ to be positive but exceeded by tᵢ such that the parameter would be in yearly units. We included case-control specific overdispersion terms θ₀ and θ1 as the CHIP carriers had greater dispersion. To adjust for batch effects, we included a random intercept, as the amount of singletons in controls varied by study.

To include the constraint on Tᵢ, we defined Tᵢ=ψᵢ*ageᵢ, ψᵢ with constrained between 0 and 1, and age, is the age at blood draw. We placed an uninformative Beta(1, 1.3) prior on ψᵢ, which is equivalent to the supposition that the driver mutation is twice as likely to be acquired in the second half of life (at the time of blood draw) then the first. We assumed the study specific deviations were exchangeable with respect to a N(0,20) prior, providing some shrinkage on the study specific intercepts. We placed a N(0,1) prior on the s, parameter to aid identification. Further details are described in the supplement.

To estimate the posterior, we used the Stan Hamiltonian monte-carlo (HMC) sampler with four separate chains, and used 400 samples of burn-in. We assessed convergence using the Rhat and effect-sample size statistics. We tried multiple parameterizations to reduce the number of divergent transitions. We performed posterior predictive checks to assess the model fit.

Simulation of HSC dynamics. We simulated the number of cells within an HSC clone as a birth-death continuous time Markov chain, which models the size of an HSC clone as the composite of simultaneous Poisson birth and Poisson death point processes. Following Watson et al., HSCs could transition to one of three states: asymmetric renewal, symmetric self-renewal, and symmetric differentiation. The rate of transition was determined by the symmetric differentiation rate of the cell per year, which was set to five. The symmetric self-renewal and symmetric differentiation increase and decrease the size of the HSC clone respectively. As asymmetric division does not affect the size of the clone, we did not explicitly simulate transition to this state. The proclivity towards self-renewal was determined by the fitness of the clone. We set the entire HSC population to acquire a single driver mutation during the ‘lifetime’ of the simulation.

Passengers were accumulated over time using a birth Poisson point process. We then calculated the number of ‘detectable’ passengers that preceded the acquisition of the driver based on whether the underlying clone had expanded to a great enough proportion of HSC cells. We examined the association between the number of detectable passengers and the fitness of the underlying HSC clone. We implemented this simulation in the Julia programming language 1.4⁶⁴ .

Re-analysis of single-cell RNA sequencing data. The cell-by-gene count matrix data for each sample from Psaila et al, generated using the 10× Genomics platform, was downloaded from Gene Expression Omnibus (GSE144568). Each matrix was loaded in Seurat with the read10× command, and only cells with a minimum of 200 features were retained using the CreateSeuratObject command. Data was log normalized using a scale factor of 10000 by the NormalizeData command. We then used the Find VariableFeatures command with ‘vst’ selection method and 2000 features. The data was scaled using ScaleData using all genes as features. We then used the RunPCA command with VariableFeatures identified earlier. For clustering, we used FindNeighbors set to the first 10 PCA dimensions and FindClusters using a resolution of 0.5. We excluded samples that did not have a distinct cluster of HSC/MPPs, defined as clusters enriched for cells that were CD34+ CD38−/lo THY1+. This left 5 healthy marrow samples (id01, id06, id09, id13, id17) and 4 MPN samples (id2, id7, id11, id14). For each of these samples, we assessed the number of cells with TCL1A transcripts within the cluster or clusters that contained HSC/MPPs, as defined above.

Additional preprocessed single-cell RNAseq from Velten et al., generated using MutaSeq, was downloaded from Gene Expression Omnibus (GSE75478) as an RDS file. We utilized data from one patient with AML (P1) and the healthy control (H1). We then determined the number of cells containing TCL1A transcript in the preleukemic ‘HSC/MPP’ and preleukemic ‘CD34+blasts and HSPCs’ clusters for the P1 sample and the ‘HSC/MPP’ cluster for the H1 sample, in both cases as defined by the original study authors.

Re-analysis of ATACseq data. We downloaded ATAC-seq data for AML samples as well as healthy controls from Corces et al. available at Gene Expression Omnibus (GSE75478). For our analysis, we used data from HSCs, defined as Lin− CD34+ CD38− CD90+ CD10− by the authors, from a healthy donor (donor7256), or preleukemic HSCs (pHSC), defined as Lin- CD34+ CD38− TIM3− CD99− by the authors. For the pHSC samples, we selected 2 where there were no detectable driver mutations in the pHSC compartment (SU336, SU306), 2 where there were DNMT3A mutations only (SU444, SU575), and 2 where there were TET2 mutations only (SU070, SU501).

Fastq files were downloaded, and ATAC-seq data analysis was performed as previously described. Briefly, reads were trimmed and filtered using fastp and mapped to the hg38 reference genome using hisat2 with the—no-spliced-alignment option. Bam files were deduplicated using Picard. Only reads mapping to chromosomes 1-22 and chrX were retained—chrY reads, mitochondrial reads, and other reads were discarded. Genome track files were created by loading the fragments for each sample into R, and exporting bigwig files normalized by reads in transcription start sites using ‘rtracklayer:export’. Coverage files were visualized using the Integrative Genomics Viewer.

CRISPR—Cas9 editing of CD34+ human HSPCs. CD34+ HSPCs from adult donors were purchased from the Cooperative Center of Excellence in Hematology (CCEH) at the Fred Hutch Cancer Research Center, Seattle, USA. TCL1A rs2887399 genotyping was performed using ThermoFisher SNP assay (Assay ID: C 15842295_20). CD34+ cells were thawed and cultured in HSC Expansion media (StemSpanll +10% CD34+ Expansion Supplement+0.1% Penicillin/Streptomycin) for 48 hours before CRISPR editing. Editing of AA VS, TET2, DNMT3A, and ASXL1 was performed by electroporation of Cas9 ribonucleoprotein complex (RNP). For each combination of rs2887399 genotype and gRNA, 100,000 cells were incubated with 3.2 ug of Synthego synthetic sgRNA guide and 8.18 ug of IDT Alt-R S.p. Cas9 Nuclease V3 for 15 minutes at room temperature before electroporation. CD34+ cells were resuspended in 18 uL of Lonza P3 solution and mixed with the ribonucleoprotein complex, and then transferred to Nucleocuvette strips for electroporation with program DZ-100 (Lonza 4D Nucleofector). Immediately following electroporation, each condition of 100,000 cells was transferred to 2 mL's of HSC Expansion media, and allowed to recover for 24 hours. CRISPR editing efficiency was measured using Sanger Sequencing and ICE Analysis.

| | | Sanger | Sanger |
|---|---|---|---|
| Tar- | Guide | Forward | Reverse |
| get | Sequence | Primer | Primer |
| AAVS | GCCAGTAGCC | GGGTCCAGGCCA | TGGCTCTTCACC |
| | AGCCCCGTCC | AGTAGGT | TTTCTAGTCCC |
| | (SEQ ID | (SEQ ID | (SEQ ID |
| | NO: 181) | NO: 182) | NO: 183) |
| TET2 | TCATGGAGCA | GGTTATGCCACA | TGACACCCCTTT |
| | TGTACTACAA | GCTTAATACAGA | AAAACTTTGG |
| | (SEQ ID | (SEQ ID | (SEQ ID |
| | NO: 184) | NO: 185) | NO: 186) |
| DNMT | GCCCGTGGGG | GGAGCTCCATCT | GGCTGGAATTGT |
| 3A | TCCGATGCTG | GAATGAGG | GTGACTTG |
| | (SEQ ID | (SEQ ID | (SEQ ID |
| | NO: 187) | NO: 188) | NO: 189) |
| ASXL1 | GTATCCGTGG | TACCCATCCCAT | GCAGCAACTGCA |
| | ACTCACCGTG | CGAATGAT | TCACAAGT |
| | (SEQ ID | (SEQ ID | (SEQ ID |
| | NO: 190) | NO: 191) | NO: 192) |

Liquid Culture Expansion Assay. 24 hours post electroporation, Lineage− CD34+ CD38− CD90+ CD45RA− cells were sorted on a BD FACS Aria III from the electroporated CD34+ cells All cells were harvested and stained with the following extracellular HSC marker panel in 100 uL of PBS+2% FBS+1 mm EDTA.

| Antibody | Vendor | Clone | Catalog# | Concentration |
|---|---|---|---|---|
| APC-CD34 | BioLegend | 561 | 343608 | 1:50 |
| PE/Cy7-CD38 | BioLegend | HIT2 | 303515 | 1:100 |
| BV421-CD90 | BioLegend | 5E10 | 562556 | 1:25 |
| BV605-CD45RA | BioLegend | HI100 | 304134 | 1:40 |
| PE/Cy5-CD2 | BioLegend | RPA-2.10 | 300210 | 1:100 |
| PE/Cy5-CD3 | BioLegend | HIT3a | 300310 | 1:100 |
| PE/Cy5-CD4 | BioLegend | SK3 | 344654 | 1:100 |
| PE/Cy5-CD8a | BioLegend | HIT8a | 300910 | 1:100 |
| PE/Cy5-CD16 | BioLegend | 3G8 | 302010 | 1:100 |
| PE/Cy5-CD19 | BioLegend | HIB19 | 302210 | 1:100 |
| PE/Cy5-CD20 | BioLegend | 2H7 | 302308 | 1:100 |
| PE/Cy5-CD56 | BioLegend | 5.1H11 | 362516 | 1:100 |
| PE/Cy5-CD235a | BioLegend | HIR2 | 306606 | 1:100 |
| PE/Cy5-CD14 | BioLegend | M5E2 | 301864 | 1:100 |
| Fixable Viability | BD | | 564997 | 1:1000 |
| Stain 700 | | | | |

4 replicates of 1,000 Lineage− CD34+ CD38− CD90+ CD45RA− cells were sorted into 100 uL of HSC Expansion media and cells were plated into a 96 well plate. The edges of 96 well plate were filled with water to keep the cultures hydrated. 4 days post sort, another 100 uL of HSC Expansion media was added to each well. 10 days post sort, the samples were transferred from the 96 well plate to a 48 well plate and an additional 400 uL of HSC Expansion media was added. 14 days post sort, the cells were harvested, and live cells were counted using trypan blue and hemocytometer. Additionally, the cells were stained with the extracellular HSC marker panel, and flow cytometry analysis was performed. Absolute number of HSC/MPPs (defined as Lin− CD34+ CD38− CD45RA−) and CD45RA^{lo} progenitors (defined as Lin−/lo CD34+ CD38− CD45RA^{lo}) were determined by multiplying the total cell count at 14 days by the percentage of cells in each compartment as determined by flow cytometry.

Flow cytometry for TCL1A staining. Anti-human TCL1A antibody clone eBio1-21 was obtained from ThermoFisher. The specificity of the antibody was assessed by staining NALM6 cells that had been CRISPR edited for TCL1A with the antibody, which confirmed only a low level of non-specific binding. To assess for TCL1A expression in cultured human HSPCs, cells in HSC Expansion media were harvested and intracellularly stained 11 days following electroporation.

| Antibody | Vendor | Clone | Catalog# | Concentration |
|---|---|---|---|---|
| e450-TCL1A | ThermoFisher | eBio1- | 48-6699- | 1 ug |
| | | 21 | 42 | |
| APC-CD34 | BioLegend | 561 | 343608 | 1:50 |
| PE/Cy7-CD38 | BioLegend | HIT2 | 303515 | 1:100 |
| FITC-CD90 | BioLegend | 5E10 | 562556 | 1:15 |
| BV605- | BioLegend | HI100 | 304134 | 1:25 |
| CD45RA | | | | |
| PE/Cy5-CD2 | BioLegend | RPA- | 300210 | 1:100 |
| | | 2.10 | | |
| PE/Cy5-CD3 | BioLegend | HIT3a | 300310 | 1:100 |
| PE/Cy5-CD4 | BioLegend | SK3 | 344654 | 1:100 |
| PE/Cy5-CD8a | BioLegend | HIT8a | 300910 | 1:100 |
| PE/Cy5-CD16 | BioLegend | 3G8 | 302010 | 1:100 |
| PE/Cy5-CD19 | BioLegend | HIB19 | 302210 | 1:100 |
| PE/Cy5-CD20 | BioLegend | 2H7 | 302308 | 1:100 |
| PE/Cy5-CD56 | BioLegend | 5.1H11 | 362516 | 1:100 |
| PE/Cy5-CD235a | BioLegend | HIR2 | 306606 | 1:100 |
| PE/Cy5-CD14 | BioLegend | M5E2 | 301864 | 1:100 |
| Fixable Viability | BD | | 564997 | 1:1000 |
| Stain 700 | | | | |

Cells were first stained with the Live/Dead and extracellular HSC markers simultaneously for 30 minutes in the dark on ice. After a PBS wash, cells were stained with 100 uL of IC Fixation Buffer for 30 minutes in the dark at room temperature. Cells were then washed twice with 1× Permeabilization Buffer. Next, cells were resuspended in 100 uL of 1× Permeabilization Buffer, and blocked with 2 uL of goat serum and 2.5 uL of TruStain FcX for 15 minutes in the dark at room temperature. Next, 1 ug of e450 antibodies (anti-TCL1A or isotype control) was added to each sample tube and stained for 30 minutes in the dark at room temperature. Cells were then washed twice with 1× Permeabilization Buffer and then resuspended in PBS before flow cytometry was performed. HSC/MPPs were defined as Lin− CD34+ CD38− CD45RA−.

ATAC-seq. 24 hours post electroporation, Lineage− CD34+ CD38− CD45RA− cells were sorted from the electroporated CD34+ cells using a BD FACS Aria III. Cells were allowed to culture for 5 days before 40,000 cells were harvested, and bulk Omni-ATAC was performed on them. Briefly, cells were lysed with ATAC-Resuspension Buffer containing 0.1% NP40, 0.1% Tween-20, and 0.01% Digitonin for 3 minutes, and then the transposition was performed for 30 minutes at 37 C using 100 nM of Illumina Tagment DNA TDE1 Enzyme and Buffer Kit per 50,000 cells . The fragmented DNA was then cleaned up using a Zymo DNA Clean and Concentrator-5 Kit (cat# D4014). The transposed fragments were amplified and indexed using NEBNext 2× Master Mix. The final PCR product was purified using the Zymo DNA Clean and Concentrator-5 Kit. Prior to sequencing, the quality of the libraries was evaluated via DNA High Sensitivity Bioanalyzer assays. The sequencing was performed using 2×75 bp reads on an Illumina NextSeq550 instrument using the High Output Kit.

ATAC-seq data analysis was performed as previously described above. Briefly, reads were trimmed and filtered using fastp and mapped to the hg38 reference genome using hisat2 with the—no-spliced-alignment option. Bam files were deduplicated using Picard. Only reads mapping to chromosomes 1-22 and chrX were retained—chrY reads, mitochondrial reads, and other reads were discarded. Genome track files were created by loading the fragments for each sample into R, and exporting bigwig files normalized by reads in transcription start sites using ‘rtracklayer:export’. Coverage files were visualized using the Integrative Genomics Viewer.

Data availability. Individual whole-genome sequence data for TOPMed whole genomes, individual-level harmonized phenotypes and the CHIP variant call sets used in this analysis are available through restricted access via the dbGaP TOPMed Exchange Area available to TOPMed investigators. Controlled-access release to the general scientific community via dbGaP is ongoing.

PACER Simulation Parameters. We assume that the accumulation of passenger mutations is described by a Poisson birth-death stochastic process. As the birth and death rates scale with the number of HSCs, we assume a linear birth-death process.

We assume that the birth rate for a given hematopoietic stem cell (HSC) i at time t with fitness sᵢ(t)) is λᵢ(t)˜Poisson(ω*Xᵢ(t)*(1+sᵢ(t))*dt), where dt represents the amount of time in years, and ω represents the number of stem cell divisions per year. We assume that the death rate can be described as

ψᵢ(t)˜Poisson(ω*Xᵢ(t)*(1−sᵢ(t))*dt

The death rate is the rate at which an HSC divides into two differentiated cells, and the birth rate is the rate at which an HSC divides into two HSCs. We don't consider asymmetric HSC differentiation as this would not change the clone size. The HSC clone cell count is defined as Xᵢ(t)=Σ_{l≤t}λᵢ(l)−ψᵢ(l), and the HSC clone size (a fraction of the total cell population) is

$${VAF}_{i} ( t ) = \frac{{X}_{i} ( t )}{{\sum }_{j} {X}_{j} ( t )} .$$

We start with 500 HSC clones, each with 200 identical cells in each clone Xᵢ(t=0)=200. Each cell divides once every three years (=⅓), and each clone with an initial sᵢ(t=0)=0. At each iteration, we also center the sᵢ(t) such that s̅ᵢ̅(̅t̅)̅=0. This means that there are 100,000 total HSCs at the start of the simulation.

For each clone, we set the passenger mutation rate:
μₚ, the passenger accumulation rate, Aᵢ(t)˜Poisson(Xᵢ(t)*μₚ*dt)
   Where Aᵢ(t) is the number of passengers accumulated in a given clone through time t. We set μₚ=0.006, which is the passenger mutation rate of a diploid genome for a single HSC per year. This implies a mutation rate of 6 passengers per year for a clone with 1000 cells, and a mutation rate of 600 passengers per year across the entire population of 100,000 HSCs. We will later consider the effects of an insensitive sequencing assay that captures a small fraction of the passengers.

We assign a single driver to one of the HSC clones, which is randomly selected among the HSC clones. The time of acquisition is uniformly drawn from each cell division after 10 years, such that are driver is equally likely to be acquired at either 10 years or 78 years. We simulate the HSC population across a lifetime of 90 years. We refer to the time of driver acquision as T_{d}.

We assume that each HSC clone can at most acquire a single driver, which represents a similar HSC population to the TOPMed CHIP driver carriers. If an HSC clone i acquires a driver at time t, we set sᵢ(t)=Beta(4,16). A Beta(4,16) random variable is bounded between 0 and 1 and has an expectation of 0.20. An HSC with ᵢ(t)=0.20 will self-renew 60% of the time, and terminally differentiate 40% of the time.

For a given HSC population, we simulate 90 years, and track the accumulation of passengers and drivers. To incorporate the censoring from using 38× sequencing coverage, we simulate whether a given passenger would be observed at 38× coverage by sampling the number of alt-reads from R˜Binomial(38,VAFᵢ(t)) and comparing R≥2, since two reads are required by our variant calling process. We refer to P(R≥2|VAF=vaf)=P(Binomial(38,vaf)≥2) We refer to the passengers that would be detected at 38× coverage as the censored founding passengers, ACᵢ(t), where t=T_{d}.

We ran the simulation 10,000 times, where at most a single HSC clone acquires a driver mutation. We then compared ACᵢ(T_{d}) to the fitness of the clone at the end of each simulation.

TABLE 3
PACER Estimates by CHIP driver gene mutation. Estimates
are relative to DNMT3A R882−, and can be interpreted as
the percentage increase in passengers after adjusting for
age at blood draw, study, and driver VAF.

| | | std. | | p. | conf. | conf. |
|---|---|---|---|---|---|---|
| term | estimate | error | statistic | value | low | high |
| U2AF1 | 1.653 | 0.097 | 5.162 | 2.45E−07 | 1.373 | 2.013 |
| SRSF2 | 1.628 | 0.055 | 8.870 | 7.33E−19 | 1.464 | 1.816 |
| JAK2 | 1.549 | 0.049 | 8.918 | 4.75E−19 | 1.409 | 1.707 |
| SF3B1 | 1.518 | 0.047 | 8.792 | 1.47E−18 | 1.385 | 1.668 |
| PPM1D | 1.334 | 0.038 | 7.505 | 6.12E−14 | 1.238 | 1.439 |
| TET2 | 1.303 | 0.021 | 12.622 | 1.59E−36 | 1.251 | 1.358 |
| ASXL1 | 1.256 | 0.029 | 7.793 | 6.56E−15 | 1.186 | 1.330 |
| GNB1 | 1.232 | 0.072 | 2.903 | 3.70E−03 | 1.073 | 1.423 |
| TP53 | 1.184 | 0.056 | 3.042 | 2.35E−03 | 1.064 | 1.323 |
| ZBTB33 | 1.117 | 0.052 | 2.107 | 3.51E−02 | 1.009 | 1.240 |
| CBL | 1.105 | 0.095 | 1.048 | 2.94E−01 | 0.921 | 1.340 |
| PDS5B | 1.084 | 0.127 | 0.637 | 5.24E−01 | 0.851 | 1.408 |
| DNMT3A | 1.058 | 0.032 | 1.764 | 7.78E−02 | 0.994 | 1.127 |
| R882+ | | | | | | |
| BRCC3 | 1.048 | 0.096 | 0.489 | 6.25E−01 | 0.873 | 1.271 |
| PHIP | 1.031 | 0.086 | 0.353 | 7.24E−01 | 0.874 | 1.227 |
| SRCAP | 1.024 | 0.078 | 0.303 | 7.62E−01 | 0.881 | 1.198 |
| GNAS | 0.971 | 0.082 | −0.362 | 7.17E−01 | 0.829 | 1.145 |
| ZNF318 | 0.945 | 0.053 | −1.074 | 2.83E−01 | 0.853 | 1.050 |
| YLPM1 | 0.921 | 0.062 | −1.331 | 1.83E−01 | 0.818 | 1.042 |

TABLE 4
Using DNMT3A as the reference, we investigated whether other genes
had different effect estimates for rs2887399. We observed that alt-
allele dosage in rs2887399 was more protective against clonal expansion
in TET2 than DNMT3A (beta = −0.24, pvalue = 9.6 × 10⁻⁴,

| | | standard | | |
|---|---|---|---|---|
| term | estimate | error | t statistic | pvalue |
| (Intercept) | −0.18 | 0.03 | −6.67 | 2.88E−11 |
| chip_driver_geneASXL1 | 0.35 | 0.07 | 4.82 | 1.53E−06 |
| chip_driver_geneJAK2 | 0.86 | 0.12 | 6.94 | 4.74E−12 |
| chip_driver_genePPM1D | 0.49 | 0.10 | 4.88 | 1.13E−06 |
| chip_driver_geneSF3B1 | 0.98 | 0.12 | 7.91 | 3.49E−15 |
| chip_driver_geneSRSF2 | 1.16 | 0.14 | 8.53 | <2e−16 |
| chip_driver_geneTET2 | 0.54 | 0.05 | 10.00 | <2e−16 |
| chip_driver_geneZNF318 | −0.18 | 0.16 | −1.15 | 0.25064 |
| rs2887399 | −0.01 | 0.03 | −0.38 | 0.70665 |
| chip_driver_geneASXL1: | 0.15 | 0.11 | 1.32 | 0.18718 |
| rs2887399 | | | | |
| chip_driver_geneJAK2: | −0.03 | 0.20 | −0.15 | 0.88208 |
| rs2887399 | | | | |
| chip_driver_genePPM1D: | 0.05 | 0.12 | 0.42 | 0.67316 |
| rs2887399 | | | | |
| chip_driver_geneSF3B1: | −0.43 | 0.19 | −2.24 | 0.02489 |
| rs2887399 | | | | |
| chip_driver_geneSRSF2: | −0.05 | 0.23 | −0.21 | 0.83119 |
| rs2887399 | | | | |
| chip_driver_geneTET2: | −0.24 | 0.07 | −3.31 | 0.00096 |
| rs2887399 | | | | |
| chip_driver_geneZNF318: | 0.08 | 0.18 | 0.48 | 0.63494 |
| rs2887399 | | | | |

TABLE 5
We performed association tests between rs2887399 and presence of a CHIP-associated
driver mutation stratified by gene. We observed that rs2887399 was associated
with significantly reduced odds of mutations in TET2, ASXL1, SF3B1, SRSF2, and
possibly JAK2. The risk reduction was particularly strong for mutations in SF3B1
and SRSF2, as well as for having >1 non-DNMT3A driver mutations.

| mutation_group | label | OR | lower_ci | upper_ci | pvalue |
|---|---|---|---|---|---|
| Multiple | 2 alt-alleles | 2.168549 | 1.441023 | 3.183366 | 3.24E−04 |
| w/DNMT3A | | | | | |
| DNMT3A | 2 alt-alleles | 1.500868 | 1.283977 | 1.74707 | 6.26E−07 |
| PPM1D | 2 alt-alleles | 1.391349 | 0.755032 | 2.388116 | 0.274741 |
| Multiple | 1 alt-allele | 1.213831 | 0.925752 | 1.587312 | 0.160105 |
| w/DNMT3A | | | | | |
| DNMT3A | 1 alt-allele | 1.198166 | 1.093194 | 1.312845 | 1.15E−04 |
| TP53 | 1 alt-allele | 1.142196 | 0.697531 | 1.864116 | 0.57217 |
| ZBTB33 | 1 alt-allele | 1.108113 | 0.619459 | 1.986318 | 0.533364 |
| SF3B1 | 1 alt-allele | 0.911671 | 0.594369 | 1.376844 | 0.663776 |
| PPM1D | 1 alt-allele | 0.879764 | 0.618726 | 1.237204 | 0.465116 |
| Multiple w/o | 1 alt-allele | 0.797023 | 0.602686 | 1.045781 | 1.02E−01 |
| DNMT3A | | | | | |
| TET2 | 1 alt-allele | 0.752596 | 0.633111 | 0.891794 | 9.66E−04 |
| ASXL1 | 1 alt-allele | 0.730255 | 0.561768 | 0.941428 | 0.014951 |
| SRSF2 | 1 alt-allele | 0.667333 | 0.588926 | 0.735203 | 0.090772 |
| TET2 | 2 alt-alleles | 0.633792 | 0.429078 | 0.903048 | 0.010516 |
| JAK2 | 2 alt-alleles | 0.598208 | 0.194008 | 1.418591 | 0.266327 |
| TP53 | 2 alt-alleles | 0.584176 | 0.1098 | 1.753191 | 0.349622 |
| JAK2 | 1 alt-allele | 0.577788 | 0.355316 | 0.908687 | 0.016913 |
| ASXL1 | 2 alt-alleles | 0.439226 | 0.202389 | 0.826649 | 0.008664 |
| ZBTB33 | 2 alt-alleles | 0.327343 | 0.015849 | 1.313609 | 0.065061 |
| SRSF2 | 2 alt-alleles | 0.325405 | 0.223008 | 0.428866 | 6.11E−02 |
| SF3B1 | 2 alt-alleles | 0.221959 | 0.025043 | 0.823681 | 0.01996 |
| Multiple w/o | 2 alt-alleles | 0.202217 | 0.056215 | 0.506556 | 1.24E−04 |
| DNMT3A | | | | | |

TABLE 6
Genotype frequencies of rs2887399 by CHIP driver gene mutation.
G is the reference allele and T is the alt allele.

| mutated_genes | count | hom_ref | het | hom_alt |
|---|---|---|---|---|
| No CHIP Drivers | 122444 | 55.16% | 37.08% | 7.76% |
| DNMT3A | 2524 | 50.94% | 39.33% | 9.73% |
| TET2 | 748 | 63.59% | 31.73% | 4.69% |
| ASXL1 | 323 | 67.91% | 29.28% | 2.80% |
| PPM1D | 192 | 61.78% | 30.89% | 7.33% |
| JAK2 | 113 | 71.17% | 25.23% | 3.60% |
| SF3B1 | 111 | 65.77% | 32.43% | 1.80% |
| TP53 | 90 | 58.43% | 39.33% | 2.25% |
| ZBTB33 | 88 | 56.82% | 40.91% | 2.27% |
| ZNF318 | 83 | 44.58% | 48.19% | 7.23% |
| DNMT3A, TET2 | 80 | 46.25% | 40.00% | 13.75% |
| SRSF2 | 79 | 72.15% | 26.58% | 1.27% |
| YLPM1 | 63 | 55.56% | 33.33% | 11.11% |
| DNMT3A, DNMT3A | 59 | 42.37% | 42.37% | 15.25% |
| GNB1 | 48 | 58.33% | 41.67% | 0.00% |
| TET2, TET2 | 47 | 70.21% | 27.66% | 2.13% |
| SRCAP | 46 | 57.78% | 35.56% | 6.67% |
| PHIP | 40 | 67.50% | 22.50% | 10.00% |
| U2AF1 | 37 | 54.05% | 35.14% | 10.81% |
| GNAS | 36 | 58.33% | 36.11% | 5.56% |
| CBL | 29 | 58.62% | 34.48% | 6.90% |
| BRCC3 | 27 | 62.96% | 33.33% | 3.70% |
| ASXL1, TET2 | 26 | 73.08% | 26.92% | 0.00% |
| PDS5B | 23 | 56.52% | 21.74% | 21.74% |
| NF1 | 21 | 57.14% | 23.81% | 19.05% |

### WORKS CITED

Steensma, D. P. et al. Clonal hematopoiesis of indeterminate potential and its distinction from myelodysplastic syndromes. Blood 126, 9-16 (2015).
Jaiswal, S. et al. Age-Related Clonal Hematopoiesis Associated with Adverse Outcomes ABSTRACT. NEJM.org. N Engl J Med 26, 2488-98 (2014). Genovese, G. et al. Clonal Hematopoiesis and Blood-Cancer Risk Inferred from Blood DNA Sequence. New England Journal of Medicine 371, 2477-2487 (2014).
Xie, M. et al. Age-related mutations associated with clonal hematopoietic expansion and malignancies. Nature Medicine 20, 1472-1478 (2014).
Abelson, S. et al. Prediction of acute myeloid leukaemia risk in healthy individuals. Nature 559, 400-404 (2018).
Desai, P. et al. Somatic mutations precede acute myeloid leukemia years before diagnosis. Nature Medicine 24, 1015-1023 (2018).
Young, A. L., Challen, G. A., Birmann, B. M. & Druley, T. E. Clonal haematopoiesis harbouring AML-associated mutations is ubiquitous in healthy adults. Nat Commun 7, 12484 (2016).
Jaiswal, S. et al. Clonal Hematopoiesis and Risk of Atherosclerotic Cardiovascular Disease. New England Journal of Medicine (2017) doi:10.1056/NEJMoa1701719.
Bick Alexander G. et al. Genetic Interleukin 6 Signaling Deficiency Attenuates Cardiovascular Risk in Clonal Hematopoiesis. Circulation 141, 124-131 (2020).
Taliun, D. et al. Sequencing of 53,831 diverse genomes from the NHLBI TOPMed Program. Nature 590, 290-299 (2021).
Bick, A. G. et al. Inherited causes of clonal haematopoiesis in 97,691 whole genomes. Nature 586, 763-768 (2020).
Cibulskis, K. et al. Sensitive detection of somatic point mutations in impure and heterogeneous cancer samples. Nature Biotechnology 31, 213-219 (2013).
Osorio, F. G. et al. Somatic Mutations Reveal Lineage Relationships and Age-Related Mutagenesis in Human Hematopoiesis. Cell Reports 25, 2308-2316.e4 (2018).
Mitchell, E. et al. Clonal dynamics of haematopoiesis across the human lifespan. 2021.08.16.456475 (2021) doi:10.1101/2021.08.16.456475.
Williams, N. et al. Phylogenetic reconstruction of myeloproliferative neoplasm reveals very early origins and lifelong evolution. 2020.11.09.374710. 11.09.374710v1 (2020) doi:10.1101/2020.11.09.374710.
Lee-Six, H. et al. Population dynamics of normal human blood inferred from somatic mutations. Nature 561, 473-478 (2018).
Fabre, M. A. et al. The longitudinal dynamics and natural history of clonal haematopoiesis. 2021.08.12.455048 (2021) doi:10.1101/2021.08.12.455048.
Alexandrov, L. B. et al. Clock-like mutational processes in human somatic cells. Nature Genetics 47, 1402-1407 (2015).
Zink, F. et al. Clonal hematopoiesis, with and without candidate driver mutations, is common in the elderly. Blood 130, 742-752 (2017).
Watson, C. J. et al. The evolutionary dynamics and fitness landscape of clonal hematopoiesis. Science 367, 1449-1454 (2020).
Deuren, R. C. van et al. Clone expansion of mutation-driven clonal hematopoiesis is associated with aging and metabolic dysfunction in individuals with obesity. 2021.05.12.443095 (2021) doi:10.1101/2021.05.12.443095.
Robertson, N. A. et al. Longitudinal dynamics of clonal hematopoiesis identifies gene-specific fitness effects. 2021.05.27.446006 (2021) doi:10.1101/2021.05.27.446006.
Zhou, W. et al. Efficiently controlling for case-control imbalance and sample relatedness in large-scale genetic association studies. Nature Genetics 50, 1335-1341 (2018).
Wang, G., Sarkar, A., Carbonetto, P. & Stephens, M. A simple new approach to variable selection in regression, with application to genetic fine mapping. Journal of the Royal Statistical Society: Series B (Statistical Methodology) 82, 1273-1300 (2020).
Dr, Z., Sp, W., N, J., T, J. & Pr, F. The ensembl regulatory build. Genome Biol 16, 56-56 (2015).
Carvalho-Silva, D. et al. Open Targets Platform: new developments and updates two years on. Nucleic Acids Res 47, D1056—D1065 (2019).
Narducci, M. G. et al. TCL1 Is Overexpressed in Patients Affected by Adult T-Cell Leukemias. Cancer Res 57, 5452-5456 (1997).
Saliba, J. et al. Germline duplication of ATG2B and GSKIP predisposes to familial myeloid malignancies. Nat Genet 47, 1131-1140 (2015).
Babushok, D. V. et al. Germline duplication of ATG2B and GSKIP genes is not required for the familial myeloid malignancy syndrome associated with the duplication of chromosome 14q32. Leukemia 32, 2720-2723 (2018).
Fishilevich, S. et al. GeneHancer: genome-wide integration of enhancers and target genes in GeneCards. Database (Oxford) 2017, (2017).
Thompson, D. J. et al. Genetic predisposition to mosaic Y chromosome loss in blood. Nature 575, 652-657 (2019).
Malcovati, L. et al. Clinical significance of somatic mutation in unexplained blood cytopenia. Blood 129, 3371-3378 (2017).
Bycroft, C. et al. Genome-wide genetic data on -500,000 UK Biobank participants. bioRxiv 166298-166298 (2017) doi:10.1101/166298.
Consortium, T. Gte. The GTEx Consortium atlas of genetic regulatory effects across human tissues. Science 369, 1318-1330 (2020).
Velten, L. et al. Identification of leukemic and pre-leukemic stem cells by clonal tracking from single-cell transcriptomics. Nat Commun 12, 1366 (2021).
Psaila, B. et al. Single-Cell Analyses Reveal Megakaryocyte-Biased Hematopoiesis in Myelofibrosis and Identify Mutant Clone-Specific Targets. Mol Cell 78, 477-492.e8 (2020).
Zhou, W. et al. Mosaic loss of chromosome Y is associated with common variation near TCL1A. Nature Genetics 48, 563-568 (2016).
Laine, J., Kunstle, G., Obata, T., Sha, M. & Noguchi, M. The Protooncogene TCL1 Is an Akt Kinase Coactivator. Molecular Cell 6, 395-407 (2000).
Lee, S. C.-W. et al. Synthetic Lethal and Convergent Biological Effects of Cancer-Associated Spliceosomal Gene Mutations. Cancer Cell 34, 225-241.e8 (2018).
Obeng, E. A. et al. Physiologic expression of SF3B1 K700E causes impaired erythropoiesis, aberrant splicing, and sensitivity to pharmacologic spliceosome modulation. Cancer Cell 30, 404-417 (2016).
Mayr, C. What Are 3′ UTRs Doing? Cold Spring Harb Perspect Biol 11, a034728 (2019).
Martincorena, I. et al. High burden and pervasive positive selection of somatic mutations in normal human skin. Science 348, 880-886 (2015).
Kakiuchi, N. & Ogawa, S. Clonal expansion in non-cancer tissues. Nature Reviews Cancer 21, 239-256 (2021).
Martincorena, I. et al. Somatic mutant clones colonize the human esophagus with age. Science 362, 911-917 (2018).
Regier, A. A. et al. Functional equivalence of genome sequencing analysis pipelines enables harmonized variant calling across human genetics projects. Nature Communications 9, 1-8 (2018).
Jun, G., Wing, M. K., Abecasis, G. R. & Kang, H. M. An efficient and scalable analysis framework for variant extraction and refinement from population scale DNA sequence data. Genome Res. gr.176552.114 (2015) doi:10.1101/gr.176552.114.
Cingolani, P. et al. A program for annotating and predicting the effects of single nucleotide polymorphisms, SnpEff: SNPs in the genome of Drosophila melanogaster strain w1118; iso-2; iso-3. Fly 6, 80-92 (2012).
Voss, K., Gentry, J. & Van der Auwera, G. Full-stack genomics pipelining with GATK4+WDL+Cromwell. in (F1000 Research, 2017). doi:10.7490/f1000research.1114631.1.
Beauchamp, E. M. et al. ZBTB33 Is Mutated in Clonal Hematopoiesis and Myelodysplastic Syndromes and Impacts RNA Splicing. Blood Cancer Discov (2021) doi:10.1158/2643-3230.BCD-20-0224.
Miller, C. A. et al. Failure to detect mutations in U2AF1 due to changes in the GRCh38 reference sequence. 2021.05.07.442430 (2021) doi:10.1101/2021.05.07.442430.
Hiatt, J. B., Pritchard, C. C., Salipante, S. J., O'Roak, B. J. & Shendure, J. Single molecule molecular inversion probes for targeted, high-accuracy detection of low-frequency variation. Genome Res. 23, 843-854 (2013).
mimips. (kitzmanlab, 2020).
Koboldt, D. C. et al. VarScan 2: Somatic mutation and copy number alteration discovery in cancer by exome sequencing. Genome Res. 22, 568-576 (2012).
Robinson, J. T. et al. Integrative genomics viewer. Nature Biotechnology 29, 24-26 (2011).
Ma, C., Blackwell, T., Boehnke, M. & Scott, L. J. Recommended Joint and Meta-Analysis Strategies for Case-Control Association Testing of Single Low-Count Variants. Genetic Epidemiology 37, 539-550 (2013).
Li, Z. et al. Dynamic Scan Procedure for Detecting Rare-Variant Association Regions in Whole-Genome Sequencing Studies. The American Journal of Human Genetics 104, 802-814 (2019).
Pedersen, B. S. & Quinlan, A. R. cyvcf2: fast, flexible variant analysis with Python. Bioinformatics 33, 1867-1869 (2017).
Bates, D. et al. Matrix: Sparse and Dense Matrix Classes and Methods. (2019). R Core Team. R: A Language and environment for statistical computing. (R Foundation for Statistical Computing, 2020).
Gogarten, S. M. et al. Genetic association testing using the GENESIS R/Bioconductor package. Bioinformatics 35, 5346-5348 (2019).
W. N. Venables & B. D. Ripley. Modern Applied Statistics with S. (Springer, 2002).
Stan Development Team. Stan Modeling Language Users Guide and Reference Manual, 2.17. (2020).
Stan Development Team. RStan: The R interface to Stan. (2020).
Bezanson, J., Edelman, A., Karpinski, S. & Shah, V. B. Julia: A fresh approach to numerical computing. (2017).
Corces, M. R. et al. Lineage-specific and single-cell chromatin accessibility charts human hematopoiesis and leukemia evolution. Nature Genetics 48, 1193-1203 (2016).
Weber, E. W. et al. Transient “rest” restores functionality in exhausted CAR-T cells via epigenetic remodeling. Science 372, eaba1786 (2021).
Omni-ATAC-seq: Improved ATAC-seq protocol. https://www.researchsquare.com (2017) doi:10.1038/protex.2017.096.
Giambartolomei, C. et al. Bayesian Test for Colocalisation between Pairs of Genetic Association Studies Using Summary Statistics. PLOS Genetics 10, e1004383 (2014).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for treating an individual for hematologic malignancies, including clonal hematopoiesis of indeterminate potential (CHIP), to reduce clonal expansion, the method comprising:
administering an effective dose of an agent that inhibits expression or activity of TCL1A.

2. The method of claim 1, wherein the agent is an anti-sense or RNAi agent.

3. The method of claim 2, wherein the agent comprises a nucleic acid sequence set forth in Table 1.

4. The method of claim 1, wherein the agent is a small molecule drug.

5. The method of claim 1, wherein the agent is a CRISPR mediated alteration of TCL1A expression.

6. The method of claim 5, wherein the CRISPR mediated alteration utilizes a guide RNA selected from the sequences set forth in Table 2.

7. The method of any of claims 1-6, wherein the individual is genotyped prior to treatment.

8. The method of claim 5, wherein the genotyping determines the alleles that are present of SNP rs2887399 and/or SNP rs11846938.

9. The method of claim 7 or claim 8, wherein the genotyping determines the presence of driver mutations in one or more of TET2, ASXL1, SF3B1, SRSF2, TP53, JAK2, PPM1 D, NRAS, KRAS, IDH1, and IDH2 prior to treatment, and found to have at least one driver mutation.

10. The method of any of claims 1-9, wherein the treatment is combined with administration of additional agents or regimens useful in the treatment of hematologic malignancies.

11. The method of any of claims 1-10, wherein the treatment provides for a reduction in the development of hematologic cancers, including without limitation acute myeloid leukemia, myelodysplastic syndrome, myeloproliferative neoplasms, chronic myeloid leukemia, chronic myelomonocytic leukemia, and diffuse large B-cell lymphoma.

12. The method of any of claims 1-10, wherein the treatment provides for a reduction in the development of heart disease.

13. A method for diagnosing or predicting clonal hematopoiesis of indeterminate potential (CHIP) in an individual, the method comprising: detecting in the individual a genetic mutation that increases TCL1 activity.

14. The method of claim 13, wherein the genotyping determines the alleles that are present of SNP rs2887399 and/or SNP rs11846938.

15. The method of claim 13 or 14, further comprising determining the presence of driver mutations in one or more of TET2, ASXL1, SF3B1, SRSF2, TP53, JAK2, PPM1 D, NRAS, KRAS, IDH1, and IDH2.

16. A method of determining the clonal growth rate of a clone from a patient sample comprising a cell population, the method comprising
sequencing the patient sample to identify mutations present in genomes of the cell population, to generate a sequence dataset;
selecting from the dataset somatic mutations that are not found in a reference set of samples sequenced with the same method to generate a set of passenger mutations;
filtering the set of passenger mutations to select somatic variants that are found only in a single genome in the dataset;
determining clonal fitness and birth date from the passenger mutation content after statistical adjustment for variant allele fraction and age of the person at time of tissue sampling.

17. The method of claim 16, further comprising filtering the set of passenger mutations to select somatic variants that are present at variant allele frequencies below the threshold for a germline variant.

18. The method of claim 16 or claim 17, further filtering the set of passenger mutations to select somatic variants having C-T and T-C variants.

19. The method of any of claims 16-18, wherein a single patient sample is used to determine clonal fitness and birth date.

20. The method of any of claims 16-19, wherein the patient sample is a hematopoietic cell population.

21. The method of claim 20, wherein the patient sample is peripheral blood.

22. The method of any of claims 16-21, wherein the steps are embodied as a program of instructions executable by computer and performed by means of software components loaded into the computer.

23. The method of any of claims 16-22, wherein an individual determined to have a high level of clonal fitness is treated in accordance with the finding.

24. The method of claim 23, wherein the individual is treated by the method of any of claims 1-12.
